(19) United States
(12) Patent Application Publication
(10) Pub. No.: US 2014/0294964 A1
(43) Pub. Date: Oct. 2, 2014

(54) COMPOSITIONS AND METHODS FOR TREATING CANCER WITH DACARBAZINE NANOEMULSIONS

(71) Applicants: Robert J. Nicolosi, Nashua, NH (US); Jean-Bosco Tagne, Brockton, MA (US)

(72) Inventors: Robert J. Nicolosi, Nashua, NH (US); Jean-Bosco Tagne, Brockton, MA (US)

(21) Appl. No.: 14/208,635

(22) Filed: Mar. 13, 2014

Related U.S. Application Data
(63) Continuation of application No. 12/309,715, filed on Mar. 29, 2010, now abandoned.
(60) Provisional application No. 60/835,186, filed on Aug. 2, 2006.

(51) Int. Cl. A61K 9/107; A61K 31/655; A61K 45/06

(52) U.S. Cl. CPC A61K 9/107; A61K 45/06; A61K 31/655
USPC 424/489; 514/151

(57) ABSTRACT

A uniform microfluidized nanoemulsion is disclosed containing an anti-cancer agent, such as dacarbazine. The microfluidized nanoemulsion improves the combination's cell membrane permeability by at least four-fold over conventional nanoemulsion compositions, which significantly increases the intracellular concentration of anticancer agents. As a nanoemulsion, dacarbazine has a greater anti-cancer efficacy than when applied as a free solution.

### FIELD OF INVENTION

The present invention relates to the field of cancer therapy. In one embodiment, the invention comprises a method to treat cancer using a uniform microfluidized nanoemulsion composition. In another embodiment, the invention relates to a composition comprising a microfluidized nanoemulsion encapsulating dacarbazine. In one embodiment, the cancer comprises a solid tumor. In one embodiment, the cancer comprises a melanoma.

### BACKGROUND OF THE INVENTION

Solid tumors arise in organs that contain stem cell populations. The tumors in these organs consist of heterogeneous populations of cancer cells that differ markedly in their ability to proliferate and form new tumors. While the majority of the cancer cells have a limited ability to divide, a population of cancer stem cells has an exclusive ability to extensively proliferate and form new tumors. Growing evidence suggests that pathways regulating a self-renewal of normal stem cells may be deregulated in cancer stem cells thereby resulting in a continuous expansion of self-renewing cancer cells and tumor formation. This suggests that agents that target the defective self-renewal pathways in cancer cells might lead to improved outcomes in the treatment of these diseases. Al-Hajj et al., “Self-renewal and solid tumor stem cells” Oncogene 23:7274-82 (2004). Currently, challenges regarding drug delivery to solid tumors are impeding progress in this field.

Drug delivery to solid tumors is one of the most challenging aspects in cancer therapy. Whereas agents seem promising during in vitro testing, clinical trials often fail due to unfavorable pharmacokinetics, poor delivery, low local concentrations, and limited accumulation in the target cell. One approach currently used in the art involves the treatment of solid tumors using tumor-associated vasculature targeting factors. These therapeutic regimens hope to reduce tumor progression by inhibiting tumor vascular development. tenHagen et al., “Solid tumor therapy: manipulation of the vasculature with TNF” Technol Cancer Res Treat 2:195-203 (2003). This approach fails, however, to directly provide a cytotoxic effect into the cancer cells themselves.

What is needed is a more effective cancer therapy method that: i) provides a composition having an improved cell membrane permeability; ii) provides an intracellular delivery of an anti-cancer agent; and iii) allows treatment of non-resectable and/or non-palpable tumors (i.e., for example, metastasized tumor cells).

### SUMMARY

The present invention relates to the field of cancer therapy. In one embodiment, the invention comprises a method to treat cancer using a uniform microfluidized nanoemulsion composition. In another embodiment, the invention relates to a composition comprising a microfluidized nanoemulsion encapsulating dacarbazine. In one embodiment, the cancer comprises a solid tumor. In one embodiment, the cancer comprises a melanoma.

In one embodiment, the present invention contemplates a uniform nanoemulsion comprising a population of particles having maximum and minimum diameters, wherein the difference between said maximum and minimum diameters does not exceed 500 nm.

In one embodiment, the present invention contemplates a uniform nanoemulsion comprising dacarbazine. In one embodiment, the nanoemulsion comprises a population of particles having diameters between approx. 30 and approx. 500 nanometers, wherein said nanoemulsion is not (or not substantially) contaminated (preferably <10%, more preferably <1%, most preferably <0.1% and even 0%) by particles having diameters larger than 500 nanometers. In one embodiment, the nanoemulsion further comprises a pharmaceutical. In one embodiment, the nanoemulsion further comprises a compound including, but not limited to, soybean oil, polysorbate 80, and HPLC grade water.

In one embodiment, the present invention contemplates a method, comprising; a) providing; i) a subject, wherein said patient exhibits at least one cancer symptom; ii) a nanoemulsion comprising dacarbazine; and b) delivering said nanoemulsion to said patients under conditions such that said nanoemulsion penetrates a cell membrane and wherein said nanoemulsion is released intracellularly. In one embodiment, the nanoemulsion comprises a uniform microfluidized nanoemulsion. In one embodiment, the nanoemulsion comprises a population of particles, wherein said particles having diameters between approximately 30 and approximately 500 nanometers, wherein said nanoemulsion is not contaminated by particles having diameters larger than 500 nanometers. In one embodiment, the cell membrane surrounds a normal cell. In one embodiment, the cell membrane surrounds a cancer cell. In one embodiment, the delivering comprises a method selected from the group consisting of intratumoral, oral, topical, transdermal, intravenous, intraperitoneal, intramuscular, and subcutaneous. In one embodiment, the nanoemulsion further comprises a pharmaceutical. In one embodiment, the nanoemulsion further comprises compound including, but not limited to, soybean oil, polysorbate 80, and HPLC grade water.

In one embodiment, the present invention contemplates a method, comprising; a) providing; i) a patient, wherein said patient is at risk for exhibiting at least one cancer symptom; ii) a nanoemulsion comprising dacarbazine; and b) delivering said nanoemulsion to said patients under conditions such that said at least one symptom is reduced. In one embodiment, the nanoemulsion comprises a uniform microfluidized nanoemulsion. In one embodiment, the nanoemulsion comprises a population of particles encapsulating said dacarbazine, wherein said particles having diameters between approximately 30 and approximately 500 nanometers, wherein said nanoemulsion is not contaminated by particles having diameters larger than 500 nanometers. In one embodiment, the cancer symptom comprises a melanoma tumor. In one embodiment, the delivering comprises a topical application. In one embodiment, the delivering comprises a method selected from the group consisting of oral, intratumoral, transdermal, intravenous, intraperitoneal, intramuscular, and subcutaneous.

In one embodiment, the present invention contemplates a method, comprising; a) providing; i) a patient, wherein said patient exhibits at least one melanoma cancer symptom; ii) a nanoemulsion comprising dacarbazine; and b) delivering said nanoemulsion to said patients under conditions such that said at least one symptom is reduced. In one embodiment, the nanoemulsion comprises a uniform microfluidized nanoemulsion. In one embodiment, the nanoemulsion comprises a population of particles encapsulating said dacarbazine, wherein said particles having diameters between approximately 30 and approximately 500 nanometers, wherein said nanoemulsion is not contaminated by particles having diameters larger than 500 nanometers. In one embodiment, the delivering comprises a topical application. In one embodiment, the delivering comprises a method selected from the group consisting of oral, intratumoral, transdermal, intravenous, intraperitoneal, intramuscular, and subcutaneous.

In one embodiment, the present invention contemplates a method, comprising; a) providing; i) a patient, wherein said patient exhibits at least one cancer symptom; ii) a uniform microfluidized nanoemulsion comprising dacarbazine; b) systemically delivering said nanoemulsion to said patients under conditions such that said at least one symptom is reduced. In one embodiment, the systemic delivery includes, but is not limited to, oral, intravenous, intraperitoneal, intramuscular, and subcutaneous. In one embodiment, the nanoemulsion further comprises an additional chemotherapeutic compound.

### DEFINITIONS

In general, the terms used herein are to be interpreted according to definitions generally accepted by those having ordinary skill in the art. Those listed below, however, are to be interpreted according to the following definitions.

The term “microfluidized”, “microfluidizing”, or “microfluidizer” as used herein refers to an instrument or a process that utilizes a continuous turbulent flow at high pressure including, but not limited to, a microfluidizer or other like device that may be useful in creating a uniform nanoemulsion. For example, microfluidizing may create a uniform nanoemulsion comprising a pharmaceutical or cosmeceutical from a premix within a thirty (30) second time frame (typically referred to a single pass exposure). Typically, a microfluidizer may be operated at a pressure of approximately 25,000 PSI to generate a uniform nanoemulsion.

The term “uniform nanoemulsion” as used herein, refers to any emulsion comprising any specified range of particle diameter sizes wherein the difference between the minimum diameter and maximum diameters do not exceed approximately 600 nm, preferably approximately 300 nm, more preferably approximately 200 nm, but most preferably approximately 100 nm (i.e., for example, microfluidization, as contemplated herein, produces a uniform nanoemulsion having a range of approximately 30-500 nm and is referred to herein as a uniform microfluidized nanoemulsion). Preferably, the total particle distribution (i.e., 100%) is encompassed within the specified range of particle diameter size. A particle diameter distribution where less than 3% is outside the specified range of particle diameter sizes is still contemplated herein as a uniform nanoemulsion.

The term “population” as used herein, refers to any mixture of nanoemulsion particles having a distribution in diameter size. For example, a population of nanoemulsion particles may range in particle diameter from between approximately 30-500 nm, preferably between approximately 35-350 nm, more preferably between approximately 40-200 nm, and even more preferably between 40-100 nm.

The term “nanoparticle” as used herein, refers to any particle having a diameter of less than 1000 nanometers (nm), or preferably less than 500 nm. These particles have sufficient internal volume such that a compound may become encapsulated during a mixing process (i.e., for example, microfluidization).

The term “compound” as used herein, refers to any pharmaceutical or cosmeceutical (i.e., for example, organic chemicals, lipids, proteins, oils, vitamins, crystals, minerals etc.) that are substantially soluble in a dispersion medium.

The term “additional chemotherapeutic compound” as used herein, refers to any pharmaceutical or cosmeceutical known to have either cytostatic or cytotoxic efficacy against cancerous cells. Other chemotherapeutic compounds include, but are not limited to, Alkeran, Cytoxan, Leukeran, Cis-platinum, BiCNU, Adriamycin, Doxorubicin, Cerubidine, Idamycin, Mithracin, Mutamycin, Fluorouracil, Methotrexate, Thioguanine, Toxotere, Etoposide, Vincristine, Irinotecan, Hycamptin, Matulane, Vumon, Hexylin, Hydroxyurea, Gemzar, Oncovin, and Etophophos.

The term “chemotherapeutic composition” as used herein, refers to any combination of additional chemotherapeutic compounds (i.e., for example, tamoxifen in combination with dacarbazine).

The term “stable” as used herein, refers to any population of nanoemulsion particles whose diameters stay within the range of approximately 30-500 nm over a prolonged period of time (i.e., for example, one (1) day to twenty-four (24) months, preferably, two (2) weeks to twelve (12) months, but more preferably two (2) months to five (5) months). For example, if a population of nanoemulsion particles is subjected to prolonged storage, temperature changes, and/or pH changes whose diameters stay within a range of between approximately 30-500 nm, the nanoemulsion is stable.

The term “pharmaceutically acceptable” as used herein, refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “pharmaceutically acceptable salts” as used herein, refers to derivatives wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

The term “therapeutically effective amount” as used herein, with respect to a drug dosage, shall mean that dosage that provides the specific pharmacological response for which the drug is administered or delivered to a significant number of subjects in need of such treatment. It is emphasized that ‘therapeutically effective amount,’ administered to a particular subject in a particular instance will not always be effective in treating the diseases described herein, even though such dosage is deemed a “therapeutically effective amount” by those skilled in the art. Specific subjects may, in fact, be “refractory” to a “therapeutically effective amount”. For example, a refractory subject may have a low bioavailability such that clinical efficacy is not obtainable. It is to be further understood that drug dosages are, in particular instances, measured as oral dosages, or with reference to drug levels as measured in blood.

The term “symptom” as used herein, refers to any subjective, objective or quantitative evidence of a disease or other physical abnormality in a subject or patient. For example, a cancer symptom may include, but is not limited to, a tumor, pain, headache, nausea etc.

The term “symptom is reduced” as used herein, refers to a qualitative or quantitative reduction in detectable symptoms, including, but not limited to, a detectable impact on the rate of recovery from disease (e.g. rate of tumor regression) or a detectable impact on the rate of development of disease (e.g., rate of tumor growth).

The term “refractory” as used herein, refers to any subject that does not respond with an expected clinical efficacy following the delivery of a compound as normally observed by practicing medical personnel.

The term “delivering” or “administering” as used herein, refers to any route for providing a pharmaceutical to a subject as accepted as standard by the medical community. For example, the present invention contemplates routes of delivering or administering that include, but are not limited to, intratumoral, oral, transdermal, intravenous, intraperitoneal, intramuscular, or subcutaneous.

The term “subject” or “patient” as used herein, refers to any animal to which an embodiment of the present invention may be delivered or administered. For example, a subject may be a human, dog, cat, cow, pig, horse, mouse, rat, gerbil, hamster etc.

The term “encapsulate”, “encapsulated”, or “encapsulating” refers to any compound that is completely surrounded by a protective material. For example, a compound may become encapsulated by a population of nanoemulsion particles formed during a mixing process (i.e., for example, microfluidization).

The term “pharmaceutical” refers to any compound, natural or synthetic, used by those having skill in the medical arts to relieve at least one symptom of an abnormal medical condition (i.e., for example, injury or disease). For example, a patient having at least one cancer symptom may be delivered an anti-cancer pharmaceutical.

The term “cell-impermeant” as used herein, refers to any compound that is not cell membrane permeable to the extent that a therapeutically-effective amount of the compound is intracellularly delivered.

The term “cell-permeant” as used herein, refers to any compound that is cell membrane permeable to the extent that a therapeutically-effective amount of the compound is intracellularly delivered.

The term “phospholipid” as used herein, refers to any compound comprising a phosphoric ester of glycerol. Alternatively, other glycerol hydroxyl groups may be esterified to fatty acids. Phospholipids may include, but are not limited to, phosphatidylcholine, phosphatidylethanolamine, phosphatidylserine, phosphatidylinositol, phosphatidylglyerol, 3′-O-lyslyphosphatidylglycerol, or diphosphatidylglycerol (cardiolipin).

The term “membrane permeability” refers to the ability of any compound (i.e., hydrophilic or hydrophobic) to pass through a phospholipid bilayer cellular membrane. Such membrane structures are common in most biological tissues including, but not limited to, epithelial cells, breast cells, nerve cells, prostate cells, kidney cells, intestinal cells, etc.

The term “at risk for” as used herein, refers to a medical condition or set of medical conditions exhibited by a patient which may predispose the patient to a particular disease or affliction. For example, these conditions may result from influences that include, but are not limited to, behavioral, emotional, chemical, biochemical, or environmental influences.

The term “cell” as used herein, refers to any small, usually microscopic, mass of protoplasm bounded externally by a semipermeable membrane, usually including one or more nuclei and various nonliving products, capable alone or interacting with other cells of performing all the fundamental functions of life, and forming the smallest structural unit of living matter capable of functioning independently. For example, a cell as contemplated herein includes, but is not limited to, an epithelial cell, a breast cell, a nerve cell, a liver cell, a lung cell, a kidney cell etc. Further, cells as contemplated herein may include, but are not limited to, normal cells (i.e., non-cancerous cells) or transformed cells (i.e., cancerous cells).

### BRIEF DESCRIPTION OF THE FIGURES

FIG. 1 presents an exemplary particle diameter distribution of one embodiment of a dacarbazine premix population.

FIG. 2 presents an exemplary particle diameter distribution of one embodiment of a microfluidized dacarbazine nanoemulsion population.

FIG. 3 presents an exemplary particle diameter distribution of one embodiment of a microfluidized cod liver oil nanoemulsion population four (4) months after preparation.

FIG. 4 presents an exemplary particle diameter distribution of one embodiment of a microfluidized tocopherol nanoemulsion population five (5) months after preparation.

FIG. 5 presents exemplary data demonstrating the improved anti-cancer efficacy of dacarbazine nanoemulsions on developing melanoma tumors.

FIG. 6 presents exemplary data demonstrating the improved anti-cancer efficacy of dacarbazine nanoemulsions melanoma tumors eight (8) weeks after administration.

FIG. 7 presents exemplary data showing a time course of δ-tocopherol plasma levels following improved membrane permeability by using a microfluidized nanoemulsion.

FIG. 8 presents exemplary data showing dynamic laser light scattering particle size analysis of nanoemulsions comprising dacarbazine (DAC) with: (a) showing the Z-average size distribution of the particle; and (b) the statistics graph measurement by model distribution.

FIG. 8A: Microfluidization-induced decrease in particle size.

FIG. 8B: Demonstrating a heterogeneity of particle size even within what appears to be a homogeneous distribution.

FIG. 9: Pattern of tumor size growth from Malme 3M xenograft mice after intramuscular (IM) or topical (TOP) administration of DAC formulations for 40 days. Rectangles: Nano-IM. Ovals: Nano-TOP. Triangle: Suspension-IM. Inverted Triangle: Suspension-TOP. Diamond: Control (empty nanoemulsion).

### DETAILED DESCRIPTION OF THE INVENTION

The present invention relates to the field of cancer therapy. In one embodiment, the invention comprises a method to treat cancer using a uniform microfluidized nanoemulsion composition. In another embodiment, the invention relates to a composition comprising a microfluidized nanoemulsion encapsulating dacarbazine. In one embodiment, the cancer comprises a solid tumor. In one embodiment, the cancer comprises a melanoma.

### I. Cancer Chemotherapy

In one embodiment, the present invention contemplates a method of treating cancer comprising providing a microfluidized nanoemulsion wherein a chemotherapeutic compound is encapsulated.

Chemotherapeutic agents can work in a number of ways. For example, chemotherapeutic can work by interfering with cell cycle progression or by generating DNA strand breaks. If the cancer cell is not able to overcome the cell cycle blockage or cell injury caused by the chemotherapeutic compound, the cell will often die via apoptotic mechanisms. The use of conventional delivery vehicles for chemotherapeutic agents in the treatment of cancer have several disadvantages. First, the cells may develop resistance to the chemotherapeutic agent because not all cells receive an initially lethal dose due to non-uniform biodistribution. Such resistance results either in the requirement for higher dosages of the drug and/or the renewed spread of the cancer. Alternatively, cellular resistance may result from biochemical metabolism of the anti-cancer agent or a functional resistance whereby the cell remains unaffected in the presence of the agent.

Conventional administration vehicles may also result in chemotherapeutic agents being toxic to the patient. Therefore, there is a practical upper limit to the amount that a patient can receive. However, when a chemotherapeutic agent is delivered by a vehicle that has enhanced cellular permeability and can be locally administered to ensure uniform biodistribution the dosage of any single drug can be lowered. This is beneficial to the patient since using lower levels of chemotherapeutic agents are generally safer for the patient. Additionally, when anti-cancer agents are delivered under conditions of enhanced membrane permeability, cancer cells are less likely to generate resistance because a greater percentage of the cancer cell will be killed upon the initial exposure.

Currently, the proportion of patients who achieve complete, prolonged (i.e., several years) remissions is believed to be below 1%. More typically, these responses are partial (i.e., 50% reduction in tumor mass) and its duration ranges from 6 to 12 months. Thus, there is still a large number of patients who do not receive objective, clinical response to these cytotoxic drugs. In some embodiments, the present invention contemplates and methods of administering a uniform microfluidized nanoemulsions comprising chemotherapeutic compositions wherein prolonged remissions occur in 5-50% of patients, preferably 15-40% of patients, and more preferably in 20-30% of patients.

Therefore, there is a need to i) improve the systemic delivery of chemotherapeutic drugs or ii) administer chemotherapeutic compositions having an equal efficacy that are less toxic than those currently administered.

The embodiments described herein as part of the present invention contemplate using a uniform nanoemulsion encapsulating a chemotherapeutic compound to reduce the symptoms of cancer and/or to induce regression of a cancer growth (i.e., for example, a tumor). Further, in some embodiments, the present invention contemplates chemotherapeutic compositions and methods using uniform microfluidized nanoemulsions for cancerous diseases, including, but not limited to, lymphomas and leukemias.

While it is within the scope of this invention that improved efficacy will be observed for any cancer, several cancer conditions are described below that may be susceptible to dacarbazine nanoemulsion therapy.

A. Melanoma

In one embodiment, the present invention contemplates a method of treating melanoma comprising providing a microfluidized nanoemulsion wherein dacarbazine, or a derivative thereof, is encapsulated.

Melanoma is one of the deadliest forms of cancer and if not detected early enough, has a high mortality rate. Melanoma can spread very rapidly but is less common than other types of skin cancer. The incidence of melanoma is steadily increasing, however, and is the leading cause of death from skin disease. For example, in the United States, 1 in 85 people will develop melanoma at some point in their life. The risk of developing melanoma increases with age, but nonetheless the disease frequently affects young, otherwise healthy people. Melanoma is the number one cause of cancer death in women aged 25-30.

Most drugs used for melanoma treatment (i.e., for example, dacarbazine) are lipid soluble (i.e., they have limited water solubility) and are often associated with significant side effects. Although it is not necessary to understand the mechanism of an invention, it is believed that the lack of water solubility results in delivery difficulties (i.e., for example, when the drug is administered to a patient).

Dacarbazine is the monotherapeutic drug of choice in the treatment of patients having metastatic melanoma without metastases to the central nervous system (CNS). Meta-analysis of 14 studies conducted between 1970 and 1994 with a total of 1167 patients with metastatic melanoma, shows an average response rate to dacarbazine alone of 18.7%, a complete remission average rate of 5.4%, and an average mean survival of 8 months. Serrone et al., “Dacarbazine-based chemotherapy for metastatic melanoma: thirty-year experience overview” J Exp Clin Cancer Res 19:21-34 (2000). In regards to long-term survival, of the 143 patients treated with dacarbazine alone, seven (7) had complete remission (5%). Among these, two (2) had remissions of 216 and 296 weeks respectively, and all other patients relapsed within one year. Hill et al., “Dimethyl triazeno imidazole carboxamide and combination therapy for melanoma. IV. Late results after complete response to chemotherapy” Cancer 53:1299-1305 (1984). Optimal response rates were obtained with a dose of 250 mg/m² of body surface administered for 5 successive days, repeated every 21 days after the last day of the treatment cycle and still is the recommended regimen. Luce et al., “Clinical trials with the antitumor agent 5-(3,3-dimethyl-1-triazeno)imidazole-4-carboxamide (NSC 45388)” Cancer Chemother Rep 54:119-24 (1970). Administration of higher doses, for example a single dose of 850 mg/m² repeated every 3 to 4 weeks, does not provide any further benefit. Montgomery J. A., “Experimental studies at Southern Research Institute with DTIC (NSC 45388)” Cancer Treat Rep 60:125-134 (1976). Administration protocols utilizing fractionated high doses (600 to 1,500 mg/m², repeated every 5 weeks) may induce fewer side effects and are less frequently associated with delayed marrow toxicity. Pritchard et al., “DTIC therapy in metastatic malignant melanoma: a simplified dose schedule” Cancer Treat Rep 64:1123-1126 (1980). There is currently no consensus on duration of therapy after complete remission. The pivotal role of dacarbazine in the treatment of metastatic melanoma thirty years after its first commercialization underscores a lack of effective therapies for this disease. D'incan et al., “Dacarbazine” Annales de Dermatologie et de Venéréologie 128(4):517-525 (2001).

Melanoma tumors comprise cells that produce melanin. When produced in normal cells, melanin is responsible for skin and hair color. Consequently, melanoma can also involve the pigmented portion of the eye. There are four (4) major types of melanoma:
i) Superficial Spreading Melanoma
This is the most common type of melanoma and is usually flat and irregular in shape and color, with varying shades of black and brown occurring at any age or in any tissue, and is most commonly in Caucasians.
ii) Nodular Melanoma
This melanoma usually starts as a dark blackish-blue or bluish-red raised area.
iii) Lentigo Maligna Melanoma
This melanoma usually occurs in the elderly and appears on sun-damaged skin of the face, neck, and arms. The affected skin areas are usually large, flat, and tan with intermixed areas of brown.
iv) Acral lentiginous melanoma
This melanoma is the least common form and usually occurs on the palms, soles, or under the nails, and is most commonly found in Black Americans.

Melanoma may appear on normal skin, or it may begin as a mole or other area that has changed in appearance. Some moles presenting at birth may develop into melanomas. The development of melanoma is related to sun exposure, particularly to sunburns during childhood, and is most common among people with fair skin, blue or green eyes, and red or blond hair.

Risk factors for developing melanoma include, but are not limited to, i) family history; ii) Red or blond hair and fair skin; iii) multiple birthmarks; iv) precancerous actinic keratoses; v) obvious freckling on the upper back; vi) three or more episodes of blistering sunburn before age 20; vii) three or more years spent at an outdoor summer job as a teenager; and ix) high levels of exposure to strong sunlight.

Symptoms of melanoma include, but are not limited to, i) a mole on the skin; ii) a sore on the skin; iii) a lump on the skin; iv) a growth on the skin; v) any changes in appearance of a pigmented skin lesion over time; vi) bleeding from a skin growth; vii) asymmetry of the affected area; viii) irregular borders or edges on the lesion or growth; ix) color variation from one area to another, with shades of tan, brown, or black (sometimes white, red, or blue) and/or a mixture of colors within a single lesion; and x) usually (but not always) larger than 6 mm in diameter.

Current treatment usually involves the surgical removal of the cancerous skin cells and a portion of the normal surrounding skin. However, only the smallest and most shallow melanomas can be cured by surgery alone. Consequently, radiation therapy, chemotherapy, and/or immunotherapy (i.e., for example, the use of medications that stimulate the immune system, such as interferon) may be recommended in addition to surgery.

Melanoma metastases may be assessed by surgical lymph node biopsy. Further, skin grafting may be necessary after the surgery if a large area of skin is affected. If the skin cancer is deeper than 4 mm or the lymph nodes have cancer, there is a high risk of the cancer spreading to other tissues and organs. Treatment with interferon after surgery may be useful for these patients. Studies have suggested that interferon improves the overall chance of cure by approximately 10%. However, interferon has many side effects and is sometimes difficult to tolerate. Melanomas that have spread beyond the skin and lymph nodes to other organs are usually not curable.

Treatment success for melanoma depends on many factors, including the patient's general health and whether the cancer has spread to the lymph nodes or other organs. If caught early, melanoma can be cured with conventional techniques. The risk of the cancer coming back increases with the depth of the tumor—deeper tumors have greater likelihood of recurring. If the cancer has spread to lymph nodes, there is a greater chance that the melanoma will come back.

B. Neuroblastoma

In one embodiment, the present invention contemplates a method of treating neuroblastoma comprising providing a microfluidized nanoemulsion wherein dacarbazine, or a derivative thereof, is encapsulated.

Several recent studies have further clarified the role of chemotherapy in newly diagnosed anaplastic glioma. For newly diagnosed glioblastoma, combined daily radiotherapy with daily temozolomide followed by six cycles of adjuvant temozolomide (a drug having a chemical structure and mechanism of action that is similar to dacarbazine) improves overall survival. This benefit is especially observed in patients with a methylated promoter of the MGMT gene which encodes an alkyltransferase; this observation however, needs confirmation. Although oligodendroglial tumors are sensitive to chemotherapy, classical adjuvant nitrosourea-based chemotherapy does not improve overall survival in newly diagnosed anaplastic oligodendroglioma, even in the subset of 1p/19q loss tumors. It may increase progression-free survival however, and further studies must show if combined modality treatment with daily chemotherapy during radiotherapy increases survival. No standard chemotherapy is currently available for highly anaplastic glioma failing first-line temozolomide-based therapy. van den Bent et al., “Recent developments in the use of chemotherapy in brain tumours” Eur J Cancer 42:582-8. Epub 2006 Jan. 20 (2006).

Neuroblastoma, the most common of all cancers found in children, may arise from a biochemical block of cellular differentiation and a resultant continuation of a proliferative state. Neuroblastoma often spontaneously reverts by undergoing partial differentiation and ultimate degeneration. In one embodiment, the present invention contemplates a useful therapeutic approach for clinical neuroblastoma comprising strategies to force neuroblastoma to differentiate.

Clinical and biologic features of this disease have been used to develop risk-based therapy approaches. Patients with low-risk disease can be treated with surgery alone. Patients with intermediate-risk features may survive after treatment with surgery and a relatively short course of standard dose chemotherapy. Unfortunately, most children with neuroblastoma present with advanced disease. More than 60% of patients with high-risk features will succumb to their disease despite intensive therapy including a myeloablative consolidation. Research efforts to understand the biologic basis of neuroblastoma and to identify new, more effective therapies are essential to improve the outcome for these children. Goldsby et al., “Neuroblastoma: evolving therapies for a disease with many faces” Paediatr Drugs. 6:107-22 (2004).

In one embodiment, the present invention contemplates a method to develop more effective neuroblastoma therapies. In one embodiment, the method comprises nude mice which are a recognized model for treatment of tumors. Although it is not necessary to understand the mechanism of an invention, it is believed that nude mice lack a fully-functional immune system and therefore do not mount a deleterious response against experimentally-induced tumors. Consequently, these mice may be a useful model system for analyses of the efficacy of anti-cancer treatments (i.e., for example, neuroblastoma solid tumors).

One known approach to treat neuroblastoma takes advantage of cell surface disialoganglioside over-expression. An immunoliposomal formulation covalently couples Fab′ fragments of the monoclonal antibody anti-GD(2) that is compatible with uptake systems in some neuroblastoma cell lines. When these immunoliposomes were loaded with either doxorubicin or the synthetic retinoid fenretinide some neuroblastoma cell proliferation inhibition was seen. Brignole et al., “Development of Fab′ fragments of anti-GD(2) immunoliposomes entrapping doxorubicin for experimental therapy of human neuroblastoma” Cancer Lett 197(1-2):199-204 (2003); and Raffaghello et al., “Immunoliposomal fenretinide: a novel antitumoral drug for human neuroblastoma” Cancer Lett 197(1-2):151-5 (2003).

Clinically, neuroblastoma usually presents as a malignant cancerous tumor in infants and children (1 out of 100,000, slightly more common in males) that develops from nerve tissue. The cause of neuroblastoma tumors is unknown. Neuroblastoma is most commonly diagnosed in children before age 5. The disorder occurs in approximately 1 out of 100,000 children and is slightly more common in boys. Neuroblastoma, however, can occur in many areas of the body and develops from the tissues that form the sympathetic nervous system (i.e., for example, exerting control over basic body functions, such as, but not limited to, heart rate, blood pressure, digestion, and levels of certain hormones). This tissue of origin for most neuroblastoma commonly begins in the abdomen from the tissues of the adrenal gland, but it may also occur in other areas. Metastasis may then involve tissues including, but not limited to, the lymph nodes, liver, bones, and bone marrow.

Commonly seen symptoms with neuroblastoma patients, include, but are not limited to, pale skin, dark circles around the eyes, chronic fatigue (i.e., for example, excessive tiredness lasting for weeks to months), diarrhea, enlarged or swollen abdomen, abdominal mass, bone pain or tenderness, difficulty breathing, malaise (i.e., for example, general discomfort or uneasiness lasting for weeks or months), flushed or red skin, profuse sweating, tachycardia, uncontrollable eye movements, paralysis of the lower extremities, uncoordinated movement, irritability, or poor temper control.

Methods of neuroblastoma diagnosis may include, but are not limited to, computed tomography (CT) scans, magnetic resonance imaging (MRI) scans, chest X-rays, bone scans, bone marrow biopsy, hormone tests (i.e., for example, epinephrine), complete blood count (CBC), urine or blood catecholamine levels, or MIBG scans.

Treatment common in the art varies depending on the location of the tumor, the extent of tumor spread and the age of the patient. In certain cases, surgery alone is enough, but often other therapies are needed. Anticancer medications (i.e., for example, chemotherapy) may be recommended if the tumor is widespread. Adjunctive radiation therapy may also be used.

The expected outcome varies. In very young children with neuroblastoma, the tumor may go away on its own, without any treatment, or the tissues of the tumor may mature and develop into a benign ganglioneuroma that can be surgically removed. In other cases, the tumor spreads rapidly. Response to treatment is variable. Treatment is often successful if the cancer has not spread, but if there has been spread to other areas, neuroblastoma is much harder to cure.

Complications may also occur during the course of neuroblastoma including, but not limited to, tumor metastasis, damage and loss of function of involved organ(s), kidney failure, liver failure, loss of blood cells produced by the bone marrow, decreased resistance to infection, or other organ system losses.

C. Breast Cancer

In one embodiment, the present invention contemplates a method of treating breast cancer comprising providing a microfluidized nanoemulsion wherein dacarbazine, or a derivative thereof, is encapsulated.

Breast cancer is a malignant growth that begins in the tissues of the breast. Over the course of a lifetime, one in eight women will be diagnosed with one of several types of breast cancer. For example, ductal carcinoma begins in the cells lining the ducts that bring milk to the nipple and accounts for more than 75% of breast cancers. Another breast cancer type, lobular carcinoma, begins in the milk-secreting glands of the breast but is otherwise fairly similar in its behavior to ductal carcinoma. Alternatively, other varieties of breast cancer can arise from the skin, fat, connective tissues, and other cells present in the breast.

In one embodiment, the present invention contemplates treating patients at risk for breast cancer. Risk factors for breast cancer include, but are not limited to, age, gender, hormonal imbalance, family history, early menstruation, and late menopause, oral contraceptives (i.e., for example, birth control pills), hormone replacement therapy, obesity, alcohol consumption, exposure to pesticides and other industrial products, diethylstilbestrol (DES), radiation, previous cancer diagnosis or strong history of cancer in the family.

In one embodiment, the present invention contemplates treating patients exhibiting symptoms of breast cancer. Symptoms for breast cancer include, but are not limited to, a breast lump or mass usually painless, firm to hard and usually with irregular borders; lump or mass in the armpit; a change in the size or shape of the breast; abnormal nipple discharge (i.e., for example, bloody, clear-to-yellow, green fluid, or purulent); changes in the color or feel of the skin of the breast, nipple, or areola; change in appearance or sensation of the nipple; unilateral breast pain, enlargement, or discomfort; bone pain, weight loss, swelling of one arm, and skin ulceration

Upon breast cancer diagnosis, additional testing is usually performed, including chest X-ray and blood tests. Various initial treatments such as, but not limited to, surgery, radiation, chemotherapy, or a combination of these may then be recommended, not only for treatment, but also to help determine the stage of disease. Breast cancer development is measured by a staging process that is important to help guide future treatment and follow-up. Breast cancer stages are currently defined as:

STAGE 0. In situ disease in which the cancerous cells are in their original location within normal breast tissue. Known as, for example, DCIS (ductoral carcinoma in situ) or LCIS (lobular carcinoma in situ) this stage represents a pre-cancerous condition, and only a small percentage of DCIS tumors develop to become invasive cancers.

STAGE I. A tumor less than 2 cm in diameter without intrabreast metastasis.

STAGE IIA. A tumor 2 to 5 cm in size without intrabreast metastasis or a tumor less than 2 cm in size with intrabreast metastasis.

STAGE IIB. A tumor greater than 5 cm in size without intrabreast metastasis or a tumor 2 to 5 cm in size with intrabreast metastasis.

STAGE IIIA. A tumor smaller than 5 cm in size with intrabreast metastasis which are attached to each other or to other structures, or tumor larger than 5 cm in size with intrabreast metastasis.

STAGE IIIB. A tumor that has penetrated outside the breast to the skin of the breast or of the chest wall or has metastasized to lymph nodes inside the chest wall along the sternum.

STAGE IV. A tumor of any size with metastases beyond the region of the breast and chest wall, such as to liver, bone, or lungs (i.e., for example, systemic metastasis).

The choice of initial breast cancer treatment may be based on more than one factor. For stage I, II, or III cancers, the main considerations are to adequately treat the cancer and prevent a recurrence either at the place of the original tumor (local) or elsewhere in the body (metastatic). For stage IV cancer, the goal is to improve symptoms and prolong survival. However, in most cases, stage IV breast cancer cannot be cured.

Hormonal therapy with tamoxifen is used to block the effects of estrogen that may otherwise help breast cancer cells to survive and grow. Most women with breast cancer tumors producing estrogen or progesterone benefit from treatment with tamoxifen. A new class of medicines called aromatase inhibitors (i.e., for example, Aromasin®) have been shown to be as good or possibly even better than tamoxifen in women with stage IV breast cancer.

Combination therapies are common treatments for many breast cancer patients. For stage 0 breast cancer, mastectomy or lumpectomy plus radiation is the standard treatment. However, there is some controversy on how best to treat DCIS. For stage I and II disease, lumpectomy (plus radiation) or mastectomy with at least “sentinel node” lymph node removal is standard treatment. Chemotherapy, hormone therapy, or both may be recommended following surgery. The presence of breast cancer in the axillary lymph nodes is very useful for staging and the appropriate follow-up treatment. Stage III patients are usually treated with surgery followed by chemotherapy with or without hormonal therapy. Radiation therapy may also be considered under special circumstances. Stage IV breast cancer may be treated with surgery, radiation, chemotherapy, hormonal therapy, or a combination of these (depending on the situation). The clinical stage of breast cancer is the best indicator for prognosis (probable outcome), in addition to some other factors. Five-year survival rates for individuals with breast cancer who receive appropriate treatment are approximately, 95% for stage 0, 88% for stage I, 66% for stage II, 36% for stage III, and 7% for stage IV.

Even with aggressive and appropriate treatments, breast cancer often spreads (metastasizes) to other parts of the body such as, but not limited to, the lungs, liver and bones. The recurrence rate is about 5% after total mastectomy and removing armpit lymph nodes when the nodes are found not to have cancer. The recurrence rate is 25% in those with similar treatment when the nodes have cancer.

Despite improvements in breast cancer diagnosis (i.e., for example, early detection), about 1-5% of women with newly diagnosed breast cancer have a distant metastasis at the time of the diagnosis. In addition, approximately 50% of the patients primarily diagnosed with only a local disease eventually relapse with metastases. Eighty-five percent (85%) of these recurrences take place within the first five years after the primary manifestation of the disease. Breast cancer metastases may be found in nearly every organ of the body at autopsy. The most common sites of metastatic involvement observed are loco-regional recurrences in the skin and soft tissues of the chest wall, as well as in axilla, and supraclavicular area. The most common sites for distant metastasis include, but are not limited to, bone (30-40%), lung, and liver.

Metastatic breast cancer is generally considered to be an incurable disease. However, the currently available treatment options often prolong the disease-free state and overall survival rate, as well as increase the quality of the life. The median survival from the manifestation of distant metastases is about three years.

In some patients, advanced disease can be controlled with therapy for many years allowing good quality of life. This is particularly evident for those patients with hormone receptor positive disease and nonvisceral sites of metastases. It is contemplated that with better understanding of the molecular factors involved in the response to chemotherapy and increased efficiency of chemotherapy, regimens will substantially extend the survival for these patients, and in some patients, perhaps even extend survival to their otherwise natural life-span. However, despite these promises, the current reality is that treatment provides only temporary control of cancer growth for most patients with metastatic breast cancer. Consequently, in one embodiment, the present invention contemplates compositions and methods to deliver chemotherapeutic compounds (i.e., for example, dacarbazine) to primary breast cancer tumors and metastases which provide more effective absorption of the chemotherapeutic compounds into a tumor cell. In order to provide the best options for treating and preventing metastases, in one embodiment, the present invention contemplates systemic administration of a uniform microfluidized nanoemulsion comprising at least one chemotherapeutic compound (i.e., for example, dacarbazine and/or tamoxifen).

Systemic drug therapy for advanced breast cancer is usually started with hormonal therapy due to its lower toxicity than the cytotoxic chemotherapies. The best candidates for hormonal therapy; based on their clinical features, are patients with a hormone receptor positive tumor (especially when both hormone receptors are positive), long term disease free survival, previous response to hormonal therapy, and non-visceral disease. Despite short second-line and even third-line responses to alternative hormonal therapies (e.g., second anti-estrogen or aromatase inhibitor) in advanced stage of breast cancer, nearly all patients finally become refractory to hormonal therapy and their disease progresses.

Due to its higher toxicity, cytotoxic chemotherapy is given to patients with disease refractory to hormonal therapy. In addition, it is frequently used as the first-line therapy for those with extensive visceral involvement of metastatic disease (e.g., lung or liver metastasis), with hormone receptor negative primary tumor, with extensive involvement of bone marrow, or with tumor that is so rapidly growing that the response to hormonal therapy can not be monitored. Combination chemotherapy for advanced breast cancer is generally considered more efficacious than single-agent therapy. In one embodiment, the present invention contemplates a uniform microfluidized nanoemulsion comprising a combination of a chemotherapeutic compounds (i.e., for example, dacarbazine and tamoxifen).

Advanced breast cancer is currently considered to be incurable and nearly all available chemotherapeutic drugs have been tested for use in its treatment. In embodiment, the present invention contemplates a uniform microfluidized nanoemulsion including, but not limited to, a chemotherapeutic compound or drug selected from the group comprising dacarbazine, anthracyclines (i.e., for example, topoll-inhibitors), doxorubicin, epirubicin, taxanes, paclitaxel, rapamycin, docetaxel, etoposide, amsacrine, and mitoxantrone.

In some embodiments, the present invention contemplates that chemotherapeutic compounds or compositions may be administered using nanoemulsions contemplated herein as adjuvant chemotherapy regimens (i.e., for example, administered in combination with either convention chemotherapy, radiotherapy or surgical intervention). For example whether given alone, or combined with other cytotoxic drugs, the objective response rate to anthracyclines generally ranges from 40% to 80% in metastatic breast cancer. Although it is not necessary to understand the mechanism of an invention, it is believed that when anthracyclines are given using a uniform microfluidized nanoemulsion the metastatic breast cancer response rate would be significantly greater than 40-80%. It is further believed that, the rate of complete response would be greater than 5-15% and improving long term remission for longer than one to two years.

In some embodiments, the present invention contemplates methods of administering chemotherapeutic compounds effective against breast cancer encapsulated within a uniform microfluidized nanoemulsion. Nanoemulsions, as contemplated herein, have been demonstrated to have improved membrane permeability. See Example 5. These nanoemulsions may be given using any route of administration including, but not limited to, oral, transdermal, intravenous, intraperitoneal, intramuscular, intratumoral, or subcutaneous. It is specifically contemplated that a systemic administration of a uniform microfluidized nanoemulsion comprising a chemotherapeutic compound effective against breast cancer (i.e., for example, dacarbazine, tamoxifen etc.) reduces the spread and growth of breast cancer metastases. Although it is not necessary to understand the mechanism of an invention, it is believed that systemically administered microfluidized nanoemulsions utilize their improved membrane permeability properties to intracellularly deliver chemotherapeutic compounds to the metastasized tumor cells. Alternatively, a local breast cancer solid tumor may received an intratumoral injection of a uniform microfluidized nanoemulsion comprising a chemotherapeutic compound.

D. Prostate Cancer

In one embodiment, the present invention contemplates a method of treating prostate cancer comprising providing a microfluidized nanoemulsion wherein dacarbazine, or a derivative thereof, is encapsulated.

Prostate cancer involves a malignant tumor growth within the prostate gland. The cause of prostate cancer is unknown, although some studies have shown a relationship between high dietary fat intake and increased testosterone levels. When testosterone levels are lowered either by surgical removal of the testicles (i.e., for example, castration or orchiectomy) or by medication, prostate cancer can regress. There is no known association with benign prostatic hyperplasia (BPH).

Prostate cancer is the third most common cause of death from cancer in men of all ages and is the most common cause of death from cancer in men over 75 years old. Prostate cancer is rarely found in men younger than 40. Men at higher risk include black men older than 60, farmers, tire plant workers, painters, and men exposed to cadmium. The lowest incidence occurs in Japanese men and vegetarians.

Prostate cancers are classified or staged based on their aggressiveness and how different they are from the surrounding prostate tissue. There are several different ways to stage tumors, a common one being the A-B-C-D staging system, also known as the Whitmore-Jewett system:

Stage A: Tumor is not palpable (not felt on physical examination), and is usually detected by accident after prostate surgery done for other reasons.

Stage B: Tumor is confined to the prostate and usually detected by physical examination or prostate specific antigen (PSA) testing.

Stage C: Tumor extends beyond the prostate capsule without spread to lymph nodes.

Stage D: Cancer has spread (metastasized) to regional lymph nodes or other parts of the body (i.e., for example, bones or lungs).

Most prostate cancers are now found before they cause symptoms because of routine PSA screening. Some likely symptoms include, but are not limited to: i) urinary hesitancy (delayed or slowed start of urinary stream); ii) urinary dribbling, especially immediately after urinating; iii) urinary retention; iii) pain with urination; iv) pain with ejaculation; v) lower back pain; vi) pain with bowel movement; vii) excessive urination at night; viii) incontinence; ix) bone pain or tenderness; x) hematuria (blood in the urine); xi) abdominal pain; xii) anemia; ivx) unintentional weight loss; and xv) lethargy.

Prostate cancer is often diagnosed using a rectal exam wherein a hard, irregular surface of an enlarged prostate is detected. Alternatively, an elevated prostate specific antigen (PSA) blood test may indicate prostate cancer. Other possible methods to diagnose prostate cancer include, but are not limited to, i) blood in urine; ii) atypical cells residing in urine or prostatic fluid (i.e., taken by biopsy); and iii) prostate biopsy cellular analysis.

Prostate cancer treatment is often controversial. For example, treatment options vary based on the stage of the tumor. In the early stages, surgical removal of the prostate (prostatectomy) and radiation therapy may be used to eradicate the tumor. Metastatic cancer of the prostate may be treated by hormonal manipulation (reducing the levels of testosterone by drugs or removal of the testes) or chemotherapy. Surgical treatment is usually only recommended as a last resort. For example, removal of prostate gland (radical prostatectomy) is often recommended for treatment of localized stage A and B prostate cancers. This is a lengthy procedure, usually performed using general or spinal anesthesia. Possible complications can include, but are not limited to, impotence and urinary incontinence.

Radiation therapy is used primarily to treat prostate cancers classified as stages A, B, or C. Whether radiation is as good as prostate removal is a debatable topic, and the decision about which to choose can be difficult. In patients whose health makes the risk of surgery unacceptably high, radiation therapy is often the preferred alternative. However, there are several side effects associated with radiation therapy—loss of appetite, fatigue, skin reactions such as redness and irritation, rectal burning or injury, diarrhea, cystitis (inflamed bladder), and blood in urine. External beam radiation therapy, for example, is usually performed 5 days a week for 6-8 weeks.

Another method for radiation therapy consists of implanting small pellets of radioactive iodine, gold, or iridium directly into the prostate tissue through a small incision. The advantage of this form of radiation therapy is that the radiation is directed at the prostate with less damage to the surrounding tissues.

Occasionally prostate cancer may be treatable using hormone therapy. Hormonal manipulation aims at lowering testosterone levels. Since prostate tumors require testosterone, reducing the testosterone level is often very effective in preventing further growth and spread of the cancer. This can be done either through surgical removal of the testes or by using medications. Hormone manipulation is mainly used to relieve symptoms in men whose cancer has spread. Preliminary evidence suggests that it may improve cure rates when combined with radiation or surgery. However, this is still under investigation.

Alternatively, synthetic drugs like Lupron® or Zoladex® that mimic the function of LHRH (luteinizing hormone releasing hormone) are being used increasingly to treat advanced prostate cancer. These medications suppress testosterone production. The procedure is often called chemical castration, because it has the same result as surgical removal of the testes, although it is reversible, unlike surgery. The drugs must be given by injection, usually every 3 months. Possible side effects include, but are not limited to, nausea and vomiting, hot flashes, anemia, lethargy, osteoporosis, reduced sexual desire, and erectile dysfunction (impotence).

Other medications used for hormonal therapy include androgen-blocking agents (i.e., for example, flutamide) which prevent testosterone from attaching to prostate cells. Possible side effects include erectile dysfunction, loss of sexual desire, liver problems, diarrhea, and enlarged breasts.

Chemotherapy is often used to treat prostate cancers that are resistant to hormonal treatments. A single drug or a combination of drugs are routinely administered in an effort to destroy cancer cells. Common medications that may be used to treat prostate cancer include, but are not limited to, mitoxantrone, prednisone, paclitaxel, docetaxel, estramustine, and adriamycin.

### II. Nanoemulsion Production Techniques

Nanoemulsions have been generated by a variety of methods. In particular, these methods provide a wide variation in particle diameter and require organic solvents and or polymers. When these known nanoemulsions are considered for an oral drug or nutrient delivery system, issues of biocompatibility and physiological side effects become an important issue.

In one embodiment, the present invention contemplates a method of making a nanoemulsion comprising a continuous turbulent flow at high pressure. In one embodiment, the high pressure turbulent flow comprises microfluidization. In one embodiment, a uniform nanoemulsion is generated from a premix using a single pass exposure (i.e., for example, within a thirty (30) second time frame). In one embodiment, the uniform nanoemulsion comprises a population of particles whose difference between the minimum and maximum diameters does not exceed approximately 500 nm. In one embodiment, a uniform nanoemulsion is generated using a pressure of at least 25,000 PSI. In one embodiment, the present invention contemplates a method of making uniform microfluidized nanoemulsions without organic solvents or polymers. In one embodiment, the microfluidized nanoemulsion is made from a suspension. In another embodiment, the microfluidized nanoemulsion is made from a microemulsion. In another embodiment, the microfluidized nanoemulsion is made from a homogenate.

In one embodiment, the present invention contemplates a uniform microfluidized nanoemulsion using compositions that are substantially soluble in a liquid dispersion medium. In one embodiment, the nanoemulsion encapsulates a composition comprising at least one compound. In one embodiment, the compositions comprise a medical formulation. In another embodiment, the compounds are selected from the group comprising dacarbazine or a pharmaceutical.

In one embodiment, the present invention contemplates a method of making a uniform microfluidized nanoemulsion comprising a population of particles whose diameter ranges from between 30-500 nm, without contamination of particle greater then 500 nm.

Microfluidization is a unique process that powers a single acting intensifier pump. The intensifier pump amplifies the hydraulic pressure to the selected level which, in turn, imparts that pressure to the product stream. As the pump travels through its pressure stroke, it drives the product at constant pressure through the interaction chamber. Within the interaction chamber are specially designed fixed-geometry microchannels through which the product stream will accelerate to high velocities, creating high shear and impact forces that generates a uniform nanoemulsion as the high velocity product stream impinges on itself and on wear-resistant surfaces.

As the intensifier pump completes its pressure stroke, it reverses direction and draws in a new volume of product. At the end of the intake stroke, it again reverses direction and drives the product at constant pressures, thereby repeating the process.

Upon exiting the interaction chamber, the product flows through an onboard heat exchanger which regulates the product to a desired temperature. At this point, the product may be recirculated through the system for further processing or directed externally to the next step in the process. Cook et al., “Apparatus For Forming Emulsions” U.S. Pat. No. 4,533,254 (1985); and Cook et al., “Method Of Forming A Microemulsion” U.S. Pat. No. 4,908,154 (1990) (both herein incorporated by reference).

Early attempts using microfluidizers to create nanoparticulate compositions required drug substances that were poorly soluble in a liquid dispersion medium. In one disclosed technology, “poorly soluble” was defined as less than 10 mg/ml. Bosch et al., “Process for preparing therapeutic compositions containing nanoparticles” U.S. Pat. No. 5,510,118 (1996)(herein incorporated by reference). While water-insolubility was preferably considered, oil-insoluble compounds were also subjected to a microfluidization process. Several others have implemented the basic '118 technology to encapsulate various insoluble compounds. In fact, these subsequent disclosures define a nanoparticle composition as “particles consisting of a poorly soluble therapeutic or diagnostic agent having adsorbed onto, or associated with, the surface thereof a non-crosslinked surface stabilizer”. Cooper et al., “Nanoparticulate Sterol Formulations And Novel Sterol Combinations” United States Patent Application Publication No. 2004/0033202 A1 (2004) (see pg 1 para 3) (herein incorporated by reference). Like the '118 patent, Cooper et al. discloses preparing nanoparticulate compositions using compounds that are poorly soluble in a liquid dispersion medium (i.e., water, oils, alcohols, glycols, etc.).

Two drugs that are insoluble in a selected liquid dispersion medium, meloxicam and topiramate, are suggested as potential candidates for improved clinical administration using the Cooper et al. nanoparticulate composition technology. Cooper et al., “Nanoparticulate meloxicam formulations” US Pat. Appln Publ. No. 2004/0229038 (2004); and Gustow et al., “Nanoparticulate topiramate formulations” US Pat. Appln Publ. No. 2004/0258758 (2004). Neither publication contains any exemplary data demonstrating the creation of a uniform microfluidized microemulsion having a particle diameter range of about 30-500 nm.

### III. Nanoemulsions as a Drug Delivery Platform

Encapsulation of therapeutic compounds for thermal, pH, or metabolic breakdown protection usually involves liposomes or other easily formed vesicles (i.e., a spontaneously forming oil-in-water emulsion). Nanoemulsions, in theory, may also provide protection for therapeutic compounds. In one embodiment, as detailed below, the present invention contemplates nanoemulsions that not only protect encapsulated compounds, but also improve intracellular drug delivery by promoting and facilitating drug transport through the plasma membrane.

In one embodiment, the present invention contemplates a premix comprising a compound substantially soluble (i.e., for example, greater than 30 mg/ml) in a liquid dispersion medium (i.e., for example, a heated liquid dispersion medium) and, optionally, an emulsifying agent including, but not limited to, phospholipids, fatty acid monoglycerides, fatty acid diglycerides, or polysorbates. In one embodiment, a nanoemulsion is created by exposing a premix to a continuous turbulent flow at a high pressure, wherein the pressure is at least 25,000 PSI. In one embodiment, the high pressure turbulent flow comprises microfluidization. In one embodiment, the nanoemulsion comprises particles encapsulating pharmaceutical formulations. In one embodiment, the nanoemulsion comprises a uniform nanoemulsion having a stable particle population. In one embodiment, the microfluidization comprises a single pass exposure (i.e., for example, approximately thirty (30) seconds). In one embodiment, a uniform microfluidized nanoemulsion comprising dacarbazine is created having an improved anti-cancer efficacy. In other embodiments, the uniform microfluidized nanoemulsion further comprises a combination of at least one conventional chemotherapeutic drug.

A. Anti-Cancer Agents Delivery

Nanoparticles i.e., nanoemulsions) consisting of paclitaxel, carmustine, camptothecin, and/or etoposide have particle population distributions ranging from 0.1-200 μm, with the “majority” of particles within the range of 0.1-1.0 μm (i.e., 100-1000 nm). Shorr et al., “Pharmaceutical And Diagnostic Compositions Containing Nanoparticles Useful For Treating Targeted Tissues And Cells” United States Patent Application Publ. No. 2005/0112207. Shorr, however, did not demonstrate that anti-cancer agent nanoemulsions have any in vivo treatment advantages over conventional anti-cancer agent compositions.

Nanoparticles containing carbamazepine, tetracaine, and prednisolone were prepared having an average particle size ranging between 91-406 nm. Muller et al., “Pharmaceutical Nanosuspensions For Medicament Administration As Systems With Increased Saturation Solubility And Rate Of Solution” U.S. Pat. No. 5,858,410. Muller et al., however, does not demonstrate an ability to create nanoemulsions having particle size distribution ranges of 30-500 nm, wherein there are no particle sizes above 500 nm. Further, no in vivo data showing that nanoemulsions have any treatment advantages over conventional therapeutic compositions is presented.

Nanoparticles of camptothecin provided particle sizes ranging from 0.2 μm wherein 99.9% of the particles were below 0.34 μm (i.e., 200-340 nm). Smaller camptothecin nanoemulsions required the addition of an osmotic pressure modifier (Trehalose) and provided particle sizes ranging from 0.070 μm, wherein 99.9% of the particles were below 0.22 μm. Sands et al., “Method For Administering Camptothecins Via Injection Of Pharmaceutical Composition Comprising Coated Particles Of A Camptothecin” U.S. Pat. No. 6,534,080. Intravenous camptothecin nanoemulsions showed improved efficacy against melanoma tumors versus the intraperitoneal injection of irinotecan, topotecan, and dacarbazine solutions. But intravenous camptothecin nanoemulsions, however, did not show improved efficacy against melanoma tumors versus orally administered camptothecin. Consequently, Sands et al. failed to show that a camptothecin nanoemulsion is more effective than a conventional oral dosage camptothecin formulation.

Microcrystalline compositions of temozolomide were reported without any particle size data. Friedman H. S., “Methods Of Using Temozolomide In the Treatment Of Cancer” U.S. Pat. No. 6,251,886. In vivo data in athymic rats induced to develop neoplastic meningitis demonstrated that temozolomide nanoemulsions were effective in improving survival time in a dose-dependent fashion. The study does not provide any comparison to conventionally administered temozolomide compositions.

Skin cancers have received some attention regarding using skin creams. Nanoemulsions containing 5-aminolevulinic acid intended for use in photodynamic therapy as well as in the photodiagnostic detection of proliferative cells have been reported. Schmid et al., “Nano-emulsion Of 5-Aminolevulinic Acid” U.S. Pat. No. 6,559,183 (2003). After homogenizing the various phases several times, the resulting 5-aminolevulinic acid particle size range was distributed between 10-200 nm. The basic nanoemulsion carrier system used in Schmid et al. requires egg lecithin (i.e., 83% phosphatidylcholine), Polysorbatum® 80, and Miglyol® 812 (a triglyceride) as previously reported. Weder et al., “Process for the production of a nanoemulsion of oil particles in an aqueous phase” U.S. Pat. No. 5,152,923 (1992) (herein incorporated by reference).

Microemulsions and nanoemulsions have been briefly mentioned as possible carriers of specific diarylchroman derivatives for the treatment of cancers such as, prostatic carcinoma, breast cancer, uterine cancer, cervical cancer, and colon cancer. Sangita et al., “(3R,4R)-Trans-3,4-diarylchroman derivatives and a method for the prevention and/or treatment of estrogen dependent diseases” United States Patent Application Publ No. 2005/0070597(herein incorporated by reference). Sangita et al. limit the technical details to liquid solutions and/or oral routes of administration and do not present any reasonable expectation of success for either making or using any microemulsion or nanoemulsion formulations.

Reduced cell proliferation and/or apoptosis in prostate cancers was seen after intratumoral injection of mycobacterial DNA suspended in a sonicated nanoparticle preparation (i.e., average particle size approximately 400 nm). Phillips et al., “Composition and method for inducing apoptosis in prostrate cancer cells” U.S. Pat. No. 6,794,368 (2004). Microfluidization techniques are only mentioned as possible (Model M-110Y, Microfluidics) and no attempts were apparently made to try this approach.

The administration of liposomes containing caroteinoids was effective in a mouse model to prevent the metastasis of M5076 reticulosarcoma. Mehta et al., “Formulation And Use of Carotenoids In Treatment Of Cancer” U.S. Pat. No. 5,811,119 (1998). The production of these liposomes required to use of organic solvents to dissolve the retinoid derivative in a phosphatidylcholine/soybean oil mixture followed by lyophilization and aqueous reconstitution.

B. Cosmetics Delivery

Nanoemulsions have been considered as potential drug delivery platforms, in many different types of formulations and compositions. For example, a nanoemulsion formulation is described that requires a surfactant mixture component wherein the mixture has two or more surfactants (usually the first having a low hydrophilic-lipophilic balance and the second having a high hydrophilic-lipophilic balance). Roessler et al., “Nanoemulsion Formulations” United States Patent Application Publ No. 2002/0155084 (herein incorporated by reference). Roessler et al. provides lengthy lists of potentially encapsulated compounds and nanoemulsion compositions. However, only compounds having specific skin permeation rates are discussed in any technical detail. Further, Roessler et al. teaches that the nanoemulsions created by the disclosed formulations form spontaneously and do not require high shear energies. Successful spontaneous formation of these nanoemulsions is dependent upon a complicated calculation involving surfactant densities and determination of the specific volume ratio's required. For example, the preferred nanoemulsion composition uses a 5:3 ratio of Span® 80 to Tween® 80 as the low and high hydrophilic-lipophilic balance surfactants, respectively.

Cosmetic formulations (i.e., those designed for topical application to the skin) are most effective when compounded as a cream, foam, or gel. These formulations are quite compatible with nanoemulsion technology. For example, dehydroepiandrosterone (DHEA) is known to be formulated as various emulsions containing various solubilizing and/or emulsifying agents. Besides the active compounds themselves, these compositions require a mixing of up to ten (10) specific ingredients that are responsible for the formation of the emulsion formulation during high pressure homogenization. Baldo et al., “Cosmetic Composition Containing A Steroid And A 2-Alkylalkanol Or An Ester Thereof” U.S. Pat. No. 6,486,147 (2002). Other simple emulsions are also described that may optionally, contain free-radical scavenger compounds in addition to the DHEA-derivatives. Dalko et al., “7-oxo-DHEA compounds for treating keratinous conditions/afflictions” U.S. Pat. No. 6,846,812 (2005).

### IV. Enhanced Nanoemulsion Membrane Permeability

One of the prerequisites for the therapeutic action of a compound is its ability to penetrate lipid cell membranes. But in order to do this the drug must generally act through its undissociated, lipid soluble moieties. This chemistry, however, conflicts with the chemistry associated with drug dissolution and its ability to be administered orally or even parenterally. Some embodiments contemplated by the present invention avoid these conflicts by encapsulating anti-cancer formulations in such a manner that also facilitate their passage through a cell membrane (i.e., for example, a tumor cell membrane or a non-tumor cell membrane).

Microemulsions have been reported as one possible carrier to address facilitated cell entry. These microemulsions are described as encapsulating hydrophobic drugs having a lipid core and stabilized by a monolayer of an amphipathic lipid (i.e., a phospholipid). Microemulsion stabilization is optimized by including a lipidized polymer that forms a matrix on the inner surface of the microparticles. Lu et al., “Artificial Lipoprotein Carrier System For Bioactive Materials” United States Patent Application Publ No. 2004/0234588. Lu et al. also describes nanoemulsions requiring a mixture of five (5) lipid components dissolved in chloroform. Following the evaporation of the organic solvent, the formulation was dissolved in a sodium chloride solution, sonicated and emulsified under pressure (70 psi, ten passes) to produce nanoparticles under 100 nm. One specific cholesterol-containing formulation utilized the cell membrane cholesterol uptake mechanism to facilitate intracellular entry of the formulation. Apparently, cholesterol transfer from the emulsion lipid core to a low-density lipoprotein (LDL) is required before this facilitated intracellular cholesterol entry occurs. Consequently, Lu et al. does not contemplate that the nanoparticles pass through a cell plasma membrane for intracellular delivery of an encapsulated drug.

The formation of a uniform mixture of predominantly small particles (i.e., for example, a population) may involve a physical process termed “emulsification”. An emulsion is traditionally defined in the art “as a system . . . consisting of a liquid dispersed with or without an emulsifier in an immiscible liquid usually in droplets of larger than colloidal size” Medline Plus Online Medical Dictionary, Merriam Webster (2005). Consequently, as the art developed emulsifiers capable of generating smaller and smaller diameter particles, the terms “microemulsion” and “nanoemulsion” became known. Conceptually, a microemulsion is one thousand-fold greater in diameter than a nanoemulsion. However, particle diameter distributions may vary widely in a non-controlled emulsification process creating considerable overlap between the nanoemulsion and microemulsion technologies.

Although it is not necessary to understand the mechanism of an invention, it is believed that a much greater surface-to-volume ratio is reached in the uniform microfluidized nanoemulsion preparations made according to the present invention (i.e., for example, up to 6 fold) and results in greater stability. Consequently, it is further believed that, any incorporated pharmaceutical has improved efficacy because of improved delivery, thereby achieving higher intracellular concentrations. It is further believed that nanoemulsion compositions, as contemplated by one embodiment of the present invention, when compared to known micron-sized micelles or microemulsions, have an improved delivery into the intracellular space of a cell because of improved cell membrane permeability (i.e., for example, a tumor cell, or an epithelial cell). See Example 5. For example, pre-formed micron-size micelles containing plant stanols were up to three (3) times more efficacious in inhibiting cholesterol absorption than a suspension of crystalline stanol. Ostlund et al., “Sitostanol administered in lecithin micelles potently reduces cholesterol absorption in humans” Am J Clin Nutr 70:826-831 (1999).

An increased in vitro carotenoid bioavailability in cell cultures was observed when solubilizing the carotenoids in micelles. Xu et al., “Solubilization and stabilization of carotenoids using micelles: delivery of lycopene to cells in culture” Lipids 34:1031-1036 (1999). A disadvantage of using micelles, however, involves the use of chlorinated organic solvents, a practice that should be avoided in the processing of medical formulations. Another in vitro experiment demonstrates that a nanoemulsion preparation of lipophilic substances, such as fatty acids, vitamins, and beta-carotene can be delivered into cell culture medium (RPMI-1640) and incorporated by TK-6 cells. Zuelli et al., “Delivering lipophilic substances into cells using nanoemulsions” U.S. Pat. No. 6,558,941 (2003) (herein incorporated by reference).

In one embodiment, the present invention contemplates a nanoemulsion produced by a continuous turbulent flow at high pressure having improved cell membrane permeability properties when compared to conventional nanoparticulate compositions and/or nanoemulsions currently known in the art. Nanoparticles are reported to deliver and/or release drugs (i.e., for example, norflaxin) and/or proteins (i.e., for example, serum albumin) more effectively than microparticles. Jeon et al., “Effect of solvent on the preparation of surfactant-free poly(DL-lactide-co-glycolide) nanoparticles and norfloxacin release characteristics’ Int J Pharm 207; 99-108 (2000); and Panyam et al., “Polymer degradation and in vitro release of a model protein from poly(D,L-lactide-co-glycolide) nano- and microparticles” J Control Release 92:173-187 (2003). One advantage of uniform microfluidized nanoemulsions over other nanoparticle preparations comprises a specific (i.e., for example, narrow) particle diameter range (i.e., for example, 30-500 nm). Most conventional nanoparticle compositions and/or nanoemulsions currently known have a wide distribution of particle diameters that interfere with the improved efficacies and membrane permeability of the smaller sized particles.

The present invention has solved the problem of generating nanoemulsions with highly variable particle diameters and provides a more uniformly small-sized nanoemulsions (i.e., for example, a uniform nanoemulsion comprising stable particles). Consequently, these uniform nanoemulsions provide improved membrane permeability when compared to conventional nanoparticle compositions and/or nanoemulsions currently known in the art independent of the mode of delivery which includes, but is not limited to, oral, transdermal, intravenous, intraperitoneal, intramuscular, intratumoral, subcutaneous, etc.

### V. Dacarbazine

Dacarbazine [5-(3,3-dimethyl-1-triazeno)imidazole-4-carboxamide] was first synthesized in 1959 at the Southern Research Institute, Birmingham, Ala. Dacarbazine was commercialized in the United States by Dome laboratories under the tradename DTIC-Dome® in the early 1970's and in France by Laboratoires Roger Bellon (subsequently Rhône-Poulenc and presently Aventis) under the trade name Dèticéne®. Marketing authorization in France (1975, revised in 1991) was obtained for metastatic melanoma, soft tissue sarcomas, Hodgkin's disease, and non-Hodgkin's lymphoma. Thirty years later dacarbazine remains the drug of choice in the treatment of melanoma. Triazenic compounds (i.e., for example, dacarbazine) comprise mono-unsaturated chains of three nitrogen atoms (triazene radical: —N═N—K<) that may be derivatized by various radicals and functional groups. The chemical formula of dacarbazine is C₆H₁₀N₆O and has a molecular weight of approximately 182.2 grams/mole.

Dacarbazine is generally believed to be physiologically inactive until activated in vivo. Kohlsmith et al., “Triazene metabolism. III. In vitro cytotoxicity towards M21 cells and in vivo antitumour activity of the proposed metabolites of the antitumour 1-aryl-3,3-dimethyltriazenes” Can J Physiol Pharmacol 62:396-402 (1983). For example, dacarbazine may be activated by hepatic metabolism, spontaneous photoactivation, and/or in the liver following microsomal demethylation catalyzed by various cytochrome P450 isoforms. Reid et al., “Metabolic activation of Dacarbazine by human cytochromes P450: the role of CYP1A1, CYP1A2 and CYP2E1” Clin Cancer Res 5:2192-7 (1999). Consequently, it is surprising to the art that the present invention contemplates, and provides data for, tumor regression and/or cancer symptom reduction following direct intratumoral dacarbazine injection.

Microsomal demethylation may occur in several stages, the first of which comprises the formation of a hydroxymethylated compound, 5-[3-hydroxymethyl-3-methyltriazenel-1yl]imidazole carboxamide (HMTIC), which may be converted into a monomethyl compound: 5-[3-methyltriazene-2-yl]imidazole-4-carboxamide (MTIC). MTIC is believed to spontaneously destabilize to form 5-aminoimidazol-me-4-carboxamide (AIC), a quantitatively principal metabolite of dacarbazine, and diazomethane. AIC can be detected 15 minutes after intravenous injection of dacarbazine. Breithaupt et al., “Pharmacokinetics of dacarbazine (DTIC) and its metabolite 5-aminoimidazole-4-carboxamide (AIC) following different doses schedules” Cancer Chemother Pharmacol 9:103-9 (1982).

Dacarbazine comprises an imidazole carboxamide derivative having structural similarities to certain purines. Although it is not necessary to understand the mechanism of an invention, it is believed that dacarbazine's primary mode of action appears to be alkylation and enhancement of nucleic acid and protein biosynthesis. For example, a methyldiazonium ion may react with a guanine of nucleic acids and forms a methylated adduct: N⁷-methylguanine (N⁷-meG) and/or O⁶-methylguanine (O⁶-meG). The N⁷-meG moiety is most prevalent and is believed responsible for G:C/C:G transversions. However, O⁶-meG is believed poorly matched with thymidine and may be responsible for coding errors thereby leading to cytotoxicity and mutagenesis. Essigmann et al., “Genetic toxicology of O⁶-methylguanine” In: Genetic toxicology of environmental chemicals: basic principles and mechanisms of action, Ramel C, Lambert B, Magnusson J Editors, Alan R. Liss, Inc, New-York, 1986, pp 433-40. Alternatively, adenine bases may be alkylated into N³-methyladenine, thereby leading to A:T/T:A transversions during DNA replication. van Delft et al., “Determination of N⁷-methylguanine DNA of white blood cells from cancer patients treated with dacarbazine” Carcinogenesis 13:12576-12579 (1992). Dacarbazine-induced alkylation produces detectable methylated adducts in circulating mononuclear cells one hour after a single injection. Souliotis et al., “In vivo formation and repair of O⁶-methylguanine in human leukocyte DNA after intravenous exposure to dacarbazine” Carcinogenesis 12:285-288 (1991).

It is further believed that dacarbazine is an effective cell cycle phase-nonspecific anti-melanoma drug. Current methods of chemotherapeutic drug (i.e., for example, dacarbazine) administration to patients, however, result in variable responses and often are associated with significant degrees of toxicity.

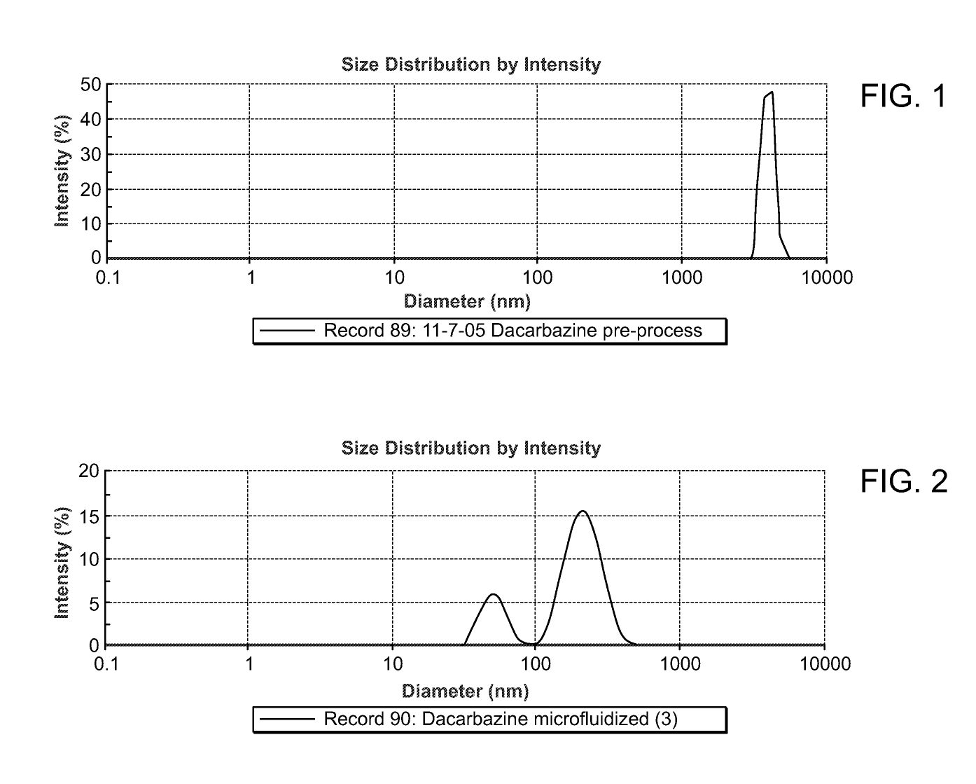

In one embodiment, the present invention contemplates a nanoemulsion preparation comprising dacarbazine, wherein said dacarbazine nanoemulsion has improved anti-cancer efficacy. In one embodiment, the nanoemulsion renders the dacarbazine water soluble. In one embodiment, the dacarbazine nanoemulsion comprises an average particle size of approximately 131 nm having a particle size distribution of approximately 30 nm-500 nm. See Table 1 and FIG. 1.

Dacarbazine was initially identified as possessing anti-cancer activity on the basis of its activity against the lymphoid cell line L1210, which was widely used for screening cytostatic drugs thirty years ago. Venditti J M., “Antitumor activity of DTIC (NSC-45388) in animals” Cancer Treat Rep 60:135-40 (1976). In mouse models receiving leukemic L1210 cells, dacarbazine administered at doses of 150-200 mg/kg prolonged survival by 43% to 67% over controls. In later clinical trials, dacarbazine was shown effective in the treatment of melanoma. Dacarbazine has variable efficacy in solid tumors in animal models as evidenced using murine cell line models including, but not limited to, melanoma B16 cells, Lewis lung carcinoma, sarcoma 180 cells, adenocarcinoma 755, lymphosarcoma P1798, Ridgeway osteogenic sarcoma, and C3H mouse mammary carcinoma, and in a Walker-256 carcinoma rat model. Dacarbazine was administered by intraperitoneal injection and its anti-tumor effect was assessed clinically by inhibition of tumor growth and by survival time. The efficacy of dacarbazine differed with the type of tumor. In the melanoma model, it did not inhibit tumor growth but did increase survival, with a more significant result when the B 16 cells were implanted subcutaneously rather than intraperitoneally (43% increased survival time versus 29%).

The above result suggested a greater efficacy of dacarbazine on skin metastases than visceral foci. The anti-metastatic effects of dacarbazine were studied by intravenously injecting melanoma B16F10 cells into syngeneic mice 24 hours before dacarbazine treatment. The number of metastatic pulmonary nodules, evaluated 14 days after inoculation, was significantly decreased (p<0.01) in animals treated with 150 mg/kg of dacarbazine compared with untreated controls. Dacarbazine is poorly absorbed in the gut and plasma peaks may vary with each oral administration wherein only 14-23% of an oral dose may be absorbed. Skibba et al., “Preliminary clinical trial and the physiologic disposition of 4(5)-(3,3-dimethyl-1-triazeno)-imidazole-5(4)-carboxamide in man” Cancer Res 29:1944-1951 (1969). Consequently, parenteral administration is currently the preferred route of administration.

The pharmacokinetics of dacarbazine and its derivatives vary considerably with the dose administered. After an intravenous injection of a typical dose of 2.65-6.85 mg/kg, serum concentration follows a biphasic curve with the first phase (T₅₀% α=2.4-3.6 min) corresponding to the elimination of 21 to 35% of the dose injected. Breithaupt et al., “Pharmacokinetics of dacarbazine (DTIC) and its metabolite 5-aminoimidazole-4-carboxamide (AIC) following different doses schedules” Cancer Chemother Pharmacol 9:103-109 (1982). The half-life of dacarbazine is 41.4 minutes. The AIC molecule forms rapidly and reaches maximal serum concentration 15 minutes after injection and then diminishes with first-order kinetics, with a half-life of 43-116 minutes and inter-individual variation off 13%. Between 40 and 50% of the dacarbazine injected and 10 to 18% of the AIC peak are excreted unmodified in the urine. Thus, 50-60% of dacarbazine intravenously injected is bioavailable.

Following injection of higher doses (850-1980 mg/m²) a similar biphasic elimination profile is observed but the T₅₀% α value is 3.5-fold greater, demonstrating the slower distribution of higher doses. The elimination half-life of AIC is also greater at high doses, as much as 1.5- to 3-fold longer than observed after injection of standard doses of dacarbazine, because of saturation in the kidneys.

Regardless of the dose injected, renal clearance of the drug exceeds creatinine clearance, indicating tubular secretion of the drug in addition to glomerular filtration. Tubular secretion is also saturated at high doses.

Dacarbazine binds weakly to proteins and diffuses into all organs, but only 13% of the injected dose crosses the blood-brain barrier. Its volume of distribution exceeds the water volume of the body, indicating an accumulation of the product in the viscera. Dacarbazine is secreted in breast milk.

The approved indications for dacarbazine, described in the French drug reference Dictionnaire Vidal, are Hodgkin's Disease, non-Hodgkin's lymphomas, and metastatic melanoma. In the United States, dacarbazine is the only single-agent chemotherapeutic (i.e., a monotherapy) approved by the FDA (excepting biological response modifiers such as interleukin-2) for treatment of disseminated melanoma. Dacarbazine has also been used in the treatment of soft tissue sarcomas and in certain neuroendocrine tumors including, but not limited to, carcinoid, pheochromocytoma, parathyroid carcinoma, or Merkel cell tumor. Bajetta et al., “5-fluorouracil, dacarbazine, and epirubicin in the treatment of patients with neuroendocrine tumors” Cancer 83:372-378 (1998).

After three decades of chemotherapeutic attempts to treat melanoma, dacarbazine remains the “gold standard”. For the most part, however, clinical trials with dacarbazine chemotherapy in melanoma have failed to show any real superiority over any other regimen. Tarhini et al., “Interleukin-2 for the treatment of melanoma” Curr Opin Investig Drugs 6:1234-1239 (2005). Overall, average response rates are <10% and the median progression-free survival is 2 months or less in contemporary trials. Clearly, there is a need to improve systemic therapy. Combination chemotherapy is associated with higher response rates than single-agent therapy but this has not translated into improved survival. An increasing number of potential therapeutic targets have been identified. Pharmacologic inhibitors are available, including but not limited to, sorafenib (BRAF inhibitor), NRAS (farnesyltransferase inhibitors), PD-0325901 (mitogen-activated protein kinase/extracellular signal-regulated kinase inhibitor), rapamycin analogues (mammalian target of rapamycin inhibitor), and agents that inhibit either vascular endothelial growth factor or its receptors. Flaherty K T, “Chemotherapy and targeted therapy combinations in advanced melanoma” Clin Cancer Res. 12(7 Pt 2):2366s-2370s (2006).

Improved long term survival was reported when a melanoma resection was followed up by successive dacarbazine-interferon administrations. In a prospective, controlled, randomized, multicenter study 252 patients with totally resected cutaneous melanoma (248 in stage II-III and 4 in stage IV) were either treated with two doses of dacarbazine (DTIC) followed by a 6-month treatment with 3 MU thrice weekly of highly purified natural interferon-alpha (n=128; arm A) or received no adjuvant treatment (n=124; arm B). After a median follow-up of 8.5 years ITT analysis showed that the difference in survival was statistically significant with respect to melanoma-related deaths and close to significance with respect to overall survival. The risk reduction of melanoma-associated death, calculated by Cox proportional hazards modeling, after adjusting for identified predictive variables, was almost 50%. The overall efficacy of the treatment appeared to be mainly attributable to effects observed in patients with deep and/or metastasizing tumours. Stadler et al., “Long-term survival benefit after adjuvant treatment of cutaneous melanoma with dacarbazine and low dose natural interferon alpha: A controlled, randomized multicoated trial” Acta Oncol. 45:389-399 (2006).

Consistent with other anti-cancer agents, dacarbazine may induce a cell (i.e., for example, a cancer cell) to undergo apoptosis. One study determined the influence of dacarbazine (DTIC) on cellular morphology and proliferation kinetics of B16 and Cloudman S91 cells using two mouse melanoma cell lines in vitro. DTIC induced morphological changes typical for apoptosis and necrosis in both cell lines. DTIC also caused cell cycle arrest in S and G2/M phase of both cell lines which showed hypertetraploidy. The highest induction of apoptosis was observed in DTIC concentration of 200 μg/ml for B16 cells (11%) and 100 μg/ml for apoptosis using Cloudman S91 cells (22.2%). Higher doses of DTIC caused an intensification of the necrotic process. Olszewska-Slonina et al., “B16 and cloudman S91 mouse melanoma cells susceptibility to apoptosis after dacarbazine treatment” Acta Pol Pharm. 62:473-483 (2005).

Conventional clinical administration regimens have been criticized for having a propensity to induce the growth of dacarbazine-resistant melanomas. The conclusions of these studies suggest that dacarbazine should be administered in combination with other chemotherapeutic drugs. For example, primary cutaneous melanoma cell lines SB2 and MeWo were repeatedly exposed in vitro to increasing concentrations of dacarbazine, and dacarbazine-resistant cell lines SB2-D and MeWo-D were selected and examined for their ability to grow and metastasize in nude mice. The dacarbazine-resistant cell lines SB2-D and MeWo-D exhibited increased tumor growth and metastatic behavior in vivo. This increase could be explained by the activation of RAF, MEK, and ERK, which led to the upregulation of IL-8 and VEGF. More IL-8, VEGF, matrix metalloproteinase-2 (MMP-2), and microvessel density (CD-31) were found in tumors produced by SB2-D and MeWo-D in vivo than in those produced by their parental counterparts. These data suggest that conventional treatment of melanoma patients with dacarbazine could select for a more aggressive melanoma phenotype. Lev et al., “Exposure of melanoma cells to dacarbazine results in enhanced tumor growth and metastasis in vivo” J Clin Oncol. 22(11):2092-2100 (2004). Epub 2004 May 3.

Predictable side effects are known to occur during conventional dacarbazine treatments. A moderate myelosuppressive effect was observed after injection of dacarbazine with fully depressed white cell and platelet counts at twenty-one to twenty-five days after treatment. This cytopenia is generally moderate, WHO grade 1 or rarely grade 2. Aplastic anemia has been observed at doses greater than 1,000 mg/m 2 per cycle

Constitutional symptoms develop in 90% of patients treated and include nausea, vomiting, and occasionally diarrhea. These occur 1 to 3 hours after the start of infusion and are generally relieved by 5-HT (serotonin) receptor-3 agonists.

Dacarbazine is usually administered intravenously and the selected dosing is dependent on whether the product is used in mono- or poly-chemotherapy, for example:
Monotherapy: 2.4 to 4.5 mg/kg/day for 4 to 5 days; additional courses may be administered after a minimal interval of 21 days after the last day of treatment;
Polytherapy: 150 to 250 mg/m²/day infused intravenously for 5 days, repeated every 3 to 4 weeks.

### Experimental

The following examples are specific embodiments as contemplated by the present invention and are not intended to be limiting.

### Example 1

### Dacarbazine Microfluidized Nanoemulsions

This example presents one dacarbazine embodiment of a microfluidized nanoemulsion. The basic step-wise procedure is as follows using the following compounds: i) dacarbazine (3×10⁻³M) MW=182.2; ii) soybean oil (Density—0.917 g/ml); iii) polysorbate 80 (Density—1.064 g/ml); and water.
1. Heat soybean oil.
2. Add dacarbazine, stir and heat 10 mins
3. Add polysorbate 80 to soybean/dacarbazine solution.
4. Heat de-ionized water to 70° C.
5. Add soybean/dacarbazine solution to heated de-ionized water, heat at 70° C. while stirring for 30 mins.
6. Homogenize Step 5 mixture for 2-4 mins
7. Stir Step 6 homogenate for 10 mins on hot plate
8. Microfluidize Step 7 homogenate using a M-110EH unit at 25,000 PSI (single pass).
9. Do particle diameter analysis using a Malvern Nano S instrument

Before microfluidization the dacarbazine mixture comprised an average particle size of approximately 3643 nm. See Table 1, FIG. 1.

TABLE 1
Particle Size Distribution of A Dacarbazine Premix

| | | | Diam. | % | Width |
|---|---|---|---|---|---|
| | | | (nm) | Intensity | (nm) |
| Z-Average(nm): | 3643 | Peak 1: | 3926 | 100 | 353.8 |
| PDI: | 0.230 | Peak 2: | 0 | 0 | 0 |
| Intercept: | 0.7052 | Peak 3: | 0 | 0 | 0 |

After microfluidization, the mean particle diameter (i.e., Peak 1/Peak 2) for the microfluidized dacarbazine nanoemulsion was 131 nm. See Table 2, FIG. 2. The average particle diameter data for the plant sterol microfluidized nanoemulsion is shown in Table 2 below.

TABLE 2
Particle Size Distribution Of A Dacarbazine Nanoemulsion

| | | | Diam. | % | Width |
|---|---|---|---|---|---|
| | | | (nm) | Intensity | (nm) |
| Z-Average: | 130.6 | Peak 1: | 221.9 | 79.05 | 59.98 |
| PDI: | 0.421 | Peak 2: | 52.82 | 20.95 | 9.832 |
| Intercept: | 0.9557 | Peak 3: | 0 | 0 | 0 |

### Example 2

### Stable Formulation of Cod Liver Oil Microfluidized Nanoemulsions

This example presents one cod liver oil embodiment of a microfluidized nanoemulsion that has a stable particle diameter for at least four months. The step-wise procedure is as follows:
1. Heat 5 g of soybean oil (65° C.)
2. Add 5 g cod liver oil, stir and heat to 80° C.
3. Add 6 g polysorbate 80, stir and heat 20 mins
4. Add 200 mL de-ionized water, stir and heat 30 mins
5. Microfluidize using a M-110EH unit once at 25,000 PSI
6. Do particle diameter analysis using a Malvern Nano S instrument

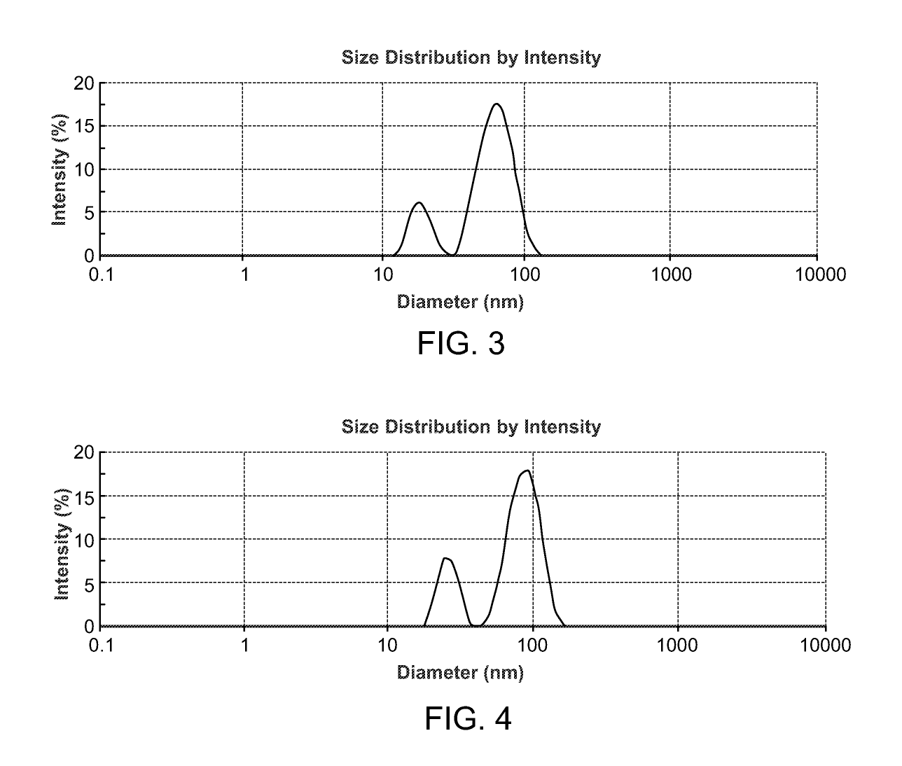

The mean particle diameter (i.e., Peak 1/Peak 2) for this cod liver oil microfluidized nanoemulsion was 58 nm (data not shown). Before microfluidization, the mean particle diameter of the cod liver oil suspension was 2,842 nm. This represents a 50-fold reduction with a single pass through the microfluidizer. Four months after the microfluidization process, the particle diameter was again determined and found not to have changed. See FIG. 3. The average particle diameter data from the four-month microfluidized sample is presented in Table 3.

TABLE 3
Microfluidized Cod Liver Oil Nanoemulsion
Four Months After Preparation

| | Diam. | % | Width |
|---|---|---|---|
| | (nm) | Intensity | (nm) |
| Peak 1: | 63.92 | 82.22 | 15.62 |
| Peak 2: | 18.51 | 17.78 | 2.771 |
| Peak 3: | 0 | 0 | 0 |

Z-Average: 45.15; PDI: 0.247; Intercept: 0.9707.

### Example 3

### Stable Formulation of Tocopherol Microfluidized Nanoemulsions

This example presents one tocopherol embodiment of a microfluidized nanoemulsion that maintains particle diameter for at least five months. The step-wise procedure is as follows:
1. Heat 13.5 g of soybean oil
2. Add 2 g tocopherol, stir and heat to 90° C.
3. Heat 2 g polysorbate 80 in 100 mL de-ionized water, heat to 75° C.
4. Add step 3 mixture to step 2 mixture
5. Heat 300 mL di-ionized water and 6 g polysorbate 80, heat till 70° C.
6. Add step 4 mixture to step 5 mixture, keep stir bar and heat on
7. Homogenize step 6 mixture for 2-4 mins
8. Stir formulation for 3-5 mins on hot plate
9. Microfluidize using a M-110EH unit once at 25,000 PSI
10. Do particle diameter analysis using a Malvern Nano S instrument

The mean particle diameter for the tocopherol microfluidized nanoemulsion was 64 nm (data not shown). Before microfluidization, the mean particle diameter for the tocopherol suspension was 1,362 nm. This represents a 21-fold reduction a single pass through the microfluidizer. Five months after the microfluidization process, the particle diameter was again determined and found not to have changed. See FIG. 4. The average particle diameter data from the five-month microfluidized sample is presented in Table 4.

TABLE 4
Microfluidized Tocopherol Nanoemulsion
Five Months After Preparation

| | Diam. | % | Width |
|---|---|---|---|
| | (nm) | Intensity | (nm) |
| Peak 1 | 88.06 | 77.84 | 19.99 |
| Peak 2 | 26.46 | 22.16 | 3.651 |
| Peak 3 | 0 | 0 | 0 |

Z-Average: 58.07; PDI: 0.234; Intercept: 0.9697

### Example 4

### Improved Anti-Cancer Efficacy of Dacarbazine Nanoemulsions

This example demonstrates that nanoemulsions comprising dacarbazine show improved efficacy over conventional administration techniques for both topical application and intramuscular injections.

Nanoemulsions were prepared according to Example 1 having the following compositions:

Nanoemulsion A (topical application):
4.2 g of soy bean oil,
4.0 g of Polysorbate® 80,
0.1 g of dacarbazine
41.7 g of H₂O
   Nanoemulsion A was then added to 50 grams of cream.

Nanoemulsion B (intramuscular injection):
2.34 g of soy bean oil
2.12 g of Tween® 80
0.051 g of dacarbazine
44.5 g of H₂O
   The Nanoemulsion B was then prepared as an injection solution having a final dacarbazine concentration of approximately 0.1 mg/50 μl.

Twenty-four (24) nude mice (average weight: 22 grams) were using in the following experimental design:
Control Group: N=4;
Nanoemulsion Control Group: N=4;
Dacarbazine Topical Cream Control Group: N=4;
Dacarbazine Intramuscular Injection Solution: N=4;
Dacarbazine Topical Cream Nanoemulsion Group: N=4; and
Dacarbazine Intramuscular Injection Nanoemulsion Group N=4.

Malme 3 M tumor cells were stored in liquid nitrogen until plating. The cells were then plated in 10% FBS and harvested for injection when reaching 90-100% confluency for injection. The confluent cells were trypsinized to detach the cells from the plate and centrifuges at 3000 rpm for 1 minute in 10% serum media to pellet the cells. The cells were then re-suspended in the necessary volume of autoclaved physiological buffered saline (1×). A stock cell suspension was prepared for injection. Before injection, a hemacytometer was used to count the number of cells per ml of the cell suspension so that 3.5×10⁵ per 100 μl cells or saline control were injected.

The cell injections (intramuscular, IM) were performed using a 1 ml syringe with a 27½ gauge needle. The area that was to be injected was cleaned with 100% alcohol first and also after the injection if any bleeding occurred. A proper cell suspension injection was verified by observing skin swelling up that disappeared after few minutes. Two (2) injection sites were used per mouse using the dorsal side of each hind leg.

After cell suspension injection the mice were monitored every twenty-four (24) hours for any signs of tumor growth. When the tumors first appear, they have the appearance and the size of a mosquito bite on the surface of the skin at the injection site. Even though all the mice were injected with the same volume and concentration of cells, the tumors appeared at different times. The tumors were also of different sizes when they first appeared (i.e., for example, 2-5 mm). As soon as the tumor became visible, a caliper was used to measure the diameter of the tumor. If the tumor was asymmetrical, the largest diameter was measured. During this time, we also checked the whole mouse for any changes in its physical well being; check other parts for sign of tumors.

All stock treatment solutions and nanoemulsions were stored at 4° C. The necessary volume was aliquoted into 2 ml syringe vials which were then allowed to warm up to room temperature before injections. Each syringe vial was labeled with the treatment name and date. For every 2 mm of tumor, 20 μl of the dacarbazine solution/nanoemulsion was injected evenly spaced adjacent to the tumor surface.

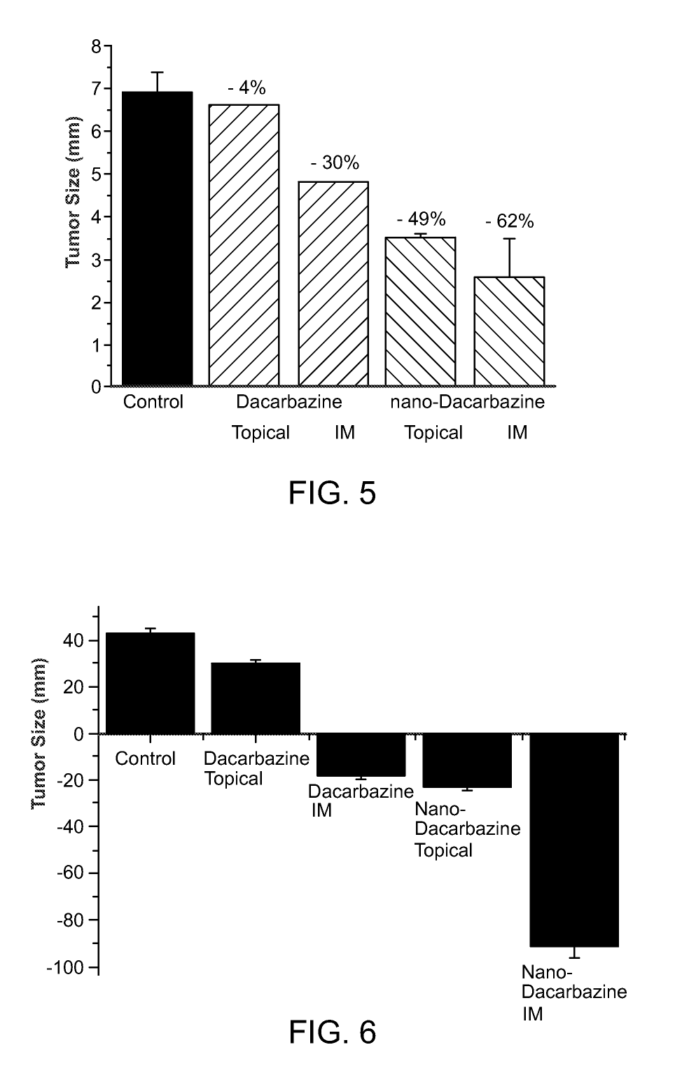

The data show that whether dacarbazine is administered as a topical cream or an intramuscular injection, the nanoemulsion preparation is more efficacious than the controls. When given topically, dacarbazine reduces tumor size from 4% (control cream) to 49% (nanoemulsion). When given as an intramuscular injection, dacarbazine reduces tumor size from 30% (control solution) to 62% (nanoemulsion). See FIG. 5.

Data collected eight (8) weeks after the treatment injections, dacarbazine nanoemulsions resulted in tumor regressions that were greater than the controls. Nanoemulsion (IM injection): 92%; Nanoemulsion (Topical): 23%; Solution (IM injection): 18%. The topical dacarbazine solution and untreated control tumors both grew in size during this same time period (30% and 43%, respectively). See FIG. 6.

### Example 5

### Improved Membrane Permeability Using Microfluidized Nanoemulsions

This example presents exemplary data showing that microfluidized nanoemulsions, as contemplated herein, substantially improves the membrane permeability of δ-tocopherol.

A microfluidized nanoemulsion containing 4.62 mg/ml δ-tocopherol was prepared according to Example 3. A non-microfluidized nanoemulsion containing 4.62 mg/ml tocopherol was prepared as follows. To 240 mls (8 oz) of water heated to 60 degrees C. for 5 minutes was added 10 g canola oil, 1.2 g of δ-tocopherol and 10 grams Tween® 80. The mixture was stirred for 20 minutes. Then 8 mls (8 g) of above mixture was mixed with 4 g of anhydrous Vanishing Creme/lotion (pharmacist grade).

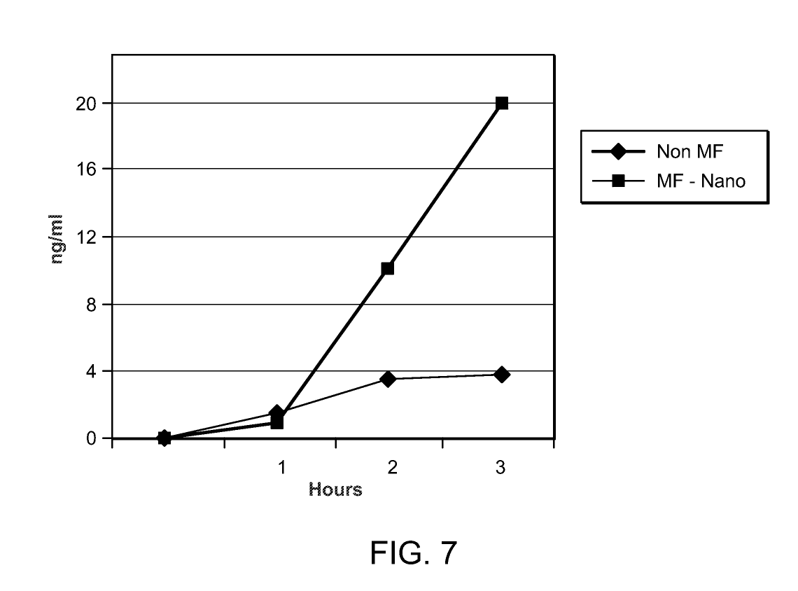

Membrane absorption was determined by applying each nanoemulsion preparation to a 1 in² shaven area on the back of a hamster (N=5). Systemic delivery of the absorbed δ-tocopherol was determined by HPCL using plasma sample extracts collected at zero time, 1 hour, 2 hours, and 3 hours post-application. These data show a progressive increase in the difference between non-microfluidized and microfluidized 5-tocopherol nanoemulsion (i.e., See Tables 5, 6, & 7, respectively and FIG. 7). The difference seen after three hours represents a 6-fold increase in membrane permeability of δ-tocopherol.

TABLE 5
δ-Tocopherol Plasma Levels After
1 Hour Of Membrane Absorption

| Treatment | Mean | Standard Error |
|---|---|---|
| Non-Micro fluidized | 1.349 | 0.488 |
| Nanoemulsion | | |
| Microfluidized | 0.859 | 0.249 |
| Nanoemulsion | | |
| Statistics | t = 0.918 df = 4 | p = 0.411 |

TABLE 6
δ-Tocopherol Plasma Levels After
2 Hours Of Membrane Absorption

| Treatment | Mean | Standard Error |
|---|---|---|
| Non-Microfluidized | 3.514 | 1.253 |
| Nanoemulsion | | |
| Microfluidized | 10.139 | 2.32 |
| Nanoemulsion | | |
| Statistics | t = −3.260 df = 4 | p = 0.031 |

TABLE 7
δ-Tocopherol Plasma Levels After
3 Hours Of Membrane Absorption

| Treatment | Mean | Standard Error |
|---|---|---|
| Non-Microfluidized | 3.833 | 1.410 |
| Nanoemulsion | | |
| Microfluidized | 20.141 | 5.341 |
| Nanoemulsion | | |
| Statistics | t = −3.793 df = 4 | p = 0.019 |

The data clearly demonstrate that microfluidized nanoemulsions, as contemplated herein, have a significantly improved membrane permeability when compared to traditional nanoemulsion preparations. Consequently, uniform microfluidized nanoemulsions have the capability of improved absorption and transfer into biological cells when compared to traditional nanoemulsion preparations.

### Example 6

### Dacarbazine Nanoemulsion Inhibition Of Xenograft Melanoma Mouse Model

This example demonstrates the acute and chronic inhibitory effect of dacarbazine nanoemulsions on mouse melanoma tumor xenographs.

Nanoemulsions were prepared according to Example 1 providing the final composition and size characteristics shown in Table 8.

TABLE 8
Particle Size Distribution Of A Dacarbazine Nanoemulsion

| | | | Diam. | % | Width |
|---|---|---|---|---|---|
| | | | (nm) | Intensity | (nm) |
| Z-Average: | 130.6 | Peak 1: | 221.9 | 79.05 | 59.98 |
| PDI: | 0.421 | Peak 2: | 52.82 | 20.95 | 9.832 |
| Intercept: | 0.9557 | Peak 3: | 0 | 0 | 0 |

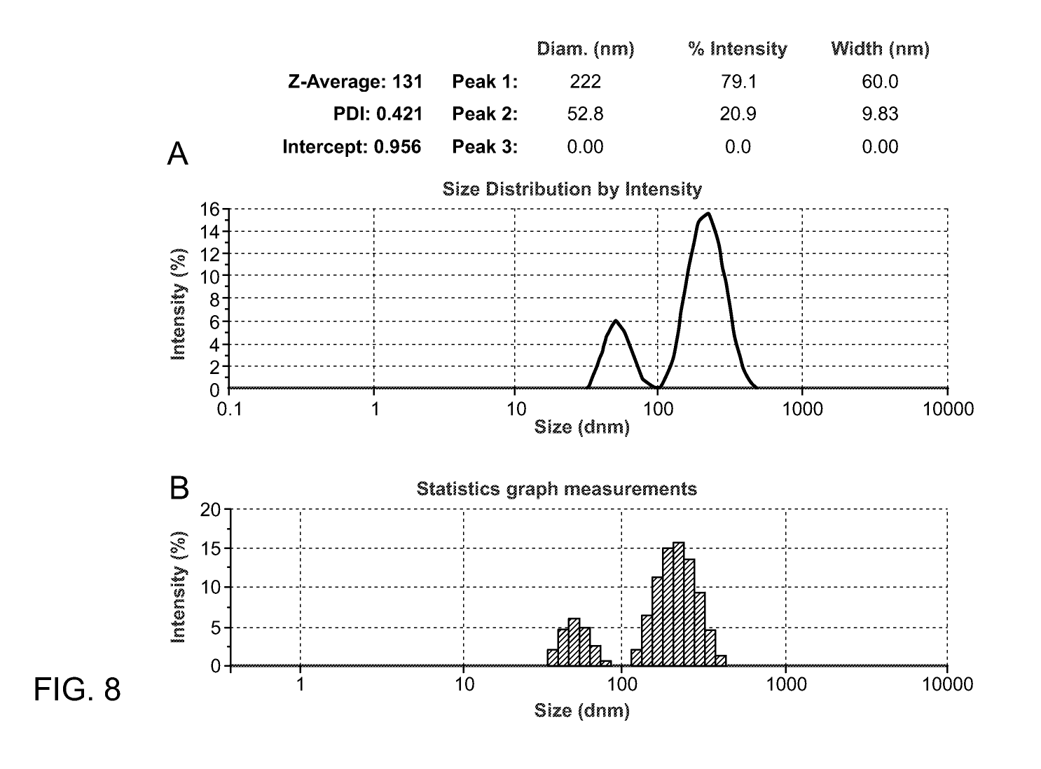

Table 8 also presents the microfluidization size characteristics that are also graphically presented in FIG. 8. After microfluidization, the mean particle diameter (i.e., Peak 1/Peak 2) for the microfluidized dacarbazine nanoemulsion was 131 nm. See Table 9, FIG. 8A. The Peak 1-Peak 2 comparison is graphically represented by histogram analysis showing the complete separation of these two peaks. Further, the histogram analysis demonstrates the narrow uniformity of each nanoemulsion population. FIG. 8B.

The experimental design involved three formulations and two modes of administration. The Control group were administered “Empty Nano-emulsion (i.e, microfluidized without dacarbazine). Two formulations containing dacarbazine were then prepared. The dacarbazine “Suspension” group (i.e., not microfludized with dacarbazine) was administered both topically (TOP) and by intramuscular injection (IM; in accordance with Example 4). The dacarbazine “Nano-Emulsion” group (i.e., microfluidized with dacarbazine) was also administered both topically (Nano-TOP) and by intramuscular injection (Nano-IM). These three formulations are characterized in Table 9.

TABLE 9
Composition, Physical, and Chemical
properties of DAC and formulations

| | | | Size | Zeta |
|---|---|---|---|---|
| Formulation | Composition | PDI | (nm) | Potential |
| Empty Nano-emulsion | SO + P80 | 0.284 | 145 | 0.304 mV |
| Suspensions | SO + P80 + DAC | 0.252 | 5470 | −89.1 mV |
| Nano-emulsion | SO + P80 + DAC | 0.421 | 131 | −40.2 mV |

SO: Soybean Oil, P80: Polysorbate 80, DAC: Dacarbazine

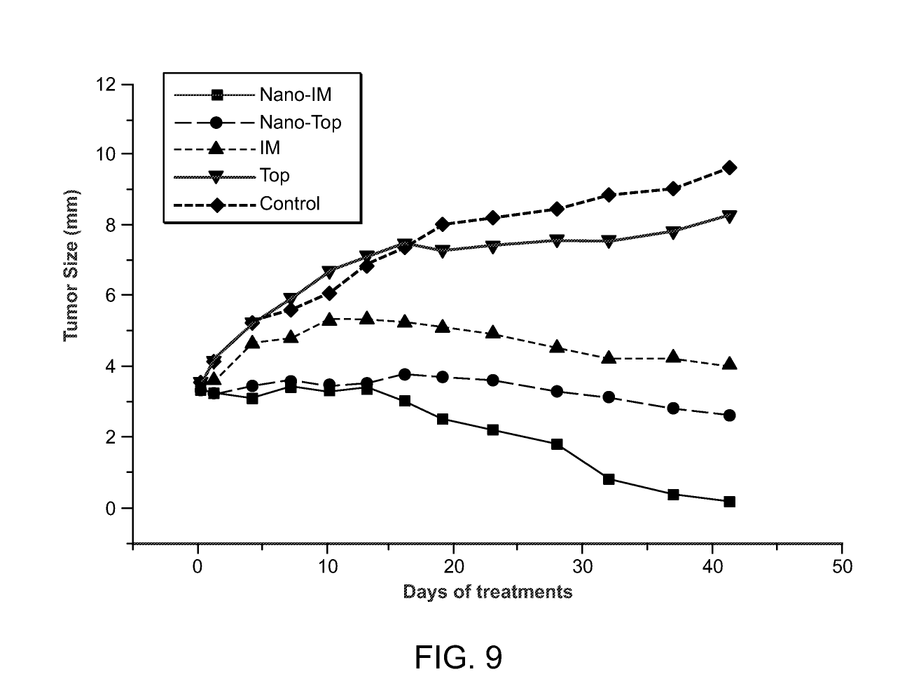

Twenty Malme 3M mice developed xenograph tumors of approximately 3.5 mm in diameter. Subsequently, each mouse was assigned to one of the above five groups (N=4, for each group) and received the appropriate treatment daily for forty (40) days. The data shows that tumor growth continued to increase in the Control and IM groups, while tumor growth either stabilized or was reduced in the TOP, Nano-IM, and Nano-TOP groups. Table 10 and FIG. 9.

TABLE 10
Effect of Different DAC Formulations on Tumor Growth in the
Xenograft Melanoma Mouse Model after 40 Days of Treatment

| | | DAC Suspensions | | DAC Nanoemulsion | |
|---|---|---|---|---|---|
| | | Route of Admistration | | | |
| Treatment Groups | Control | IM | TOP | EM | TOP |
| Tumor size increase | 6.9 ± 0.5 | 6.6 ± 0.2 | 4.8 ± 0.2 | 3.5 ± 0.1 | 2.7 ± 0.3 |
| (mm) | | | | | |
| % decrease from | | −4% ^{a} | −30% ^{b} | −49% ^{c} | −61% ^{d} |
| Control | | | | | |

| | | |
|---|---|---|
| % decrease of IM | −27% ^{e} | −23% ^{e} |
| relative to TOP | | |
| within each group | | |

Values represent Mean ± SEM. Different superscripts indicate p at least < 0.05 difference from each other.

The mouse groups were followed for another twelve weeks after the forty day drug administration period and monitored for additional tumor growth. The data shows that growth continued in the Control, IM but not the TOP, Nano-IM, and Nano-TOP groups. Table 11.

TABLE 11
Effect of Different DAC Formulations on Tumor Growth in the Xenograft
Melanoma Mouse Model after 12 Weeks of Cessation of Drug Treatment

| | | DAC Suspensions | | DAC Nanoemulsion | |
|---|---|---|---|---|---|
| | | Route of Admistration | | | |
| Treatment Groups | Control | IM | TOP | IM | TOP |
| Tumor size increase | 9.9 ± 0.7 ^{a} | 8.5 ± 0.5 | 3.9 ± 0.2 | 2.7 ± 0.1 | 0.2 ± 0.1 |
| (mm) | | | | | |
| % decrease from | | −14% ^{a} | −61% ^{b} | −73% ^{c} | −98% ^{d} |
| Control | | | | | |

| | | |
|---|---|---|
| % decrease of IM | −54% ^{e} | −93% ^{e} |
| relative to TOP | | |
| within each group | | |

Values represent Mean ± SEM. Different superscripts indicate p at least < 0.05 difference from each other.

1-5. (canceled)

6. A method, comprising:
a) providing:
i) a subject exhibiting at least one cancer symptom;
ii) a nanoemulsion composition comprising decarbazine; and,
b) administering said nanoemulsion composition to said subject under conditions such that said nanoemulsion composition penetrates a cell membrane and wherein said decarbazine is released intracellularly,
wherein the nanoemulsion composition comprises a uniform population of particles having diameters between about 30 and about 500 nanometers and not contaminated by particles having diameters larger than 500 nanometers.

7.-8. (canceled)

9. The method of claim 6, wherein said cell membrane surrounds a normal cell, a cancer cell, a cancer stem cell, or any combination thereof.

10. (canceled)

11. The method of claim 6, wherein the administering comprises a method selected from the group consisting of intratumoral, oral, topical, transdermal, intravenous, intraperitoneal, intramuscular, and subcutaneous.

12. (canceled)

13. The method of claim 6, wherein said nanoemulsion further comprises soybean oil, polysorbate 80, HPLC grade water, and any combination thereof.

14. The method of claim 6,
wherein the step of administering achieves delivering said nanoemulsion in an amount sufficient to reduce at least one cancer symptom.

15. The method of claim 14, wherein said nanoemulsion comprises a uniform microfluidized nanoemulsion.

16. (canceled)

17. The method of claim 14, wherein said cancer symptom comprises a melanoma tumor or a solid tumor.

18.-26. (canceled)

27. The method of claim 6, wherein said nanoemulsion further comprises an additional pharmaceutical.

28. The method of claim 27, wherein the additional pharmaceutical is a chemotherapeutic compound.
